(19) United States
(12) Patent Application Publication
(10) Pub. No.: US 2023/0126920 A1
(43) Pub. Date: Apr. 27, 2023

(54) METHOD AND DEVICE FOR CLASSIFICATION OF URINE SEDIMENT GENOMIC DNA, AND USE OF URINE SEDIMENT GENOMIC DNA

(71) Applicants: Beijing Institute of Genomics, Chinese Academy of Sciences (China National Center for Bioinformation, Beijing (CN); Peking University First Hospital, Beijing (CN)

(72) Inventors: Weimin Ci, (US); Zhengzheng Xu, (US); Liqun Zhou, (US)

(73) Assignees: Beijing Institute of Genomics, Chinese Academy of Sciences (China National Center for Bioinformation, Beijing (CN); Peking University First Hospital, Beijing (CN)

(21) Appl. No.: 17/755,721

(22) PCT Filed: Oct. 22, 2020
(86) PCT No.: PCT/CN2020/122821

§ 371 Date: Nov. 21, 2022

(30) Foreign Application Priority Data
Nov. 8, 2019 (CN) 201911088433.6

(51) Int. Cl. C12Q 1/6886; G16B 20/00; G16B 40/20

(52) U.S. Cl. CPC C12Q 1/6886; G16B 20/00; G16B 40/20; C12Q 2600/156; C12Q 2600/154

(57) ABSTRACT

The present invention relates to a DNA classification method, comprising calculating the MHL value of a DNA methylation haplotype block and/or the DNA copy number variation data of a sample of interest; calculating the similarity between the MHL value of the DNA methylation haplotype block of the sample of interest DNA and the MHL value of a DNA methylation haplotype region of a respective classification label, and/or the similarity between the copy number variation data of the sample of interest DNA and the DNA copy number variation data of a respective classification label; and determining a classification for the DNA in the sample of interest by using a classifier model and based on the similarity. The present invention provides new means with good specificity and sensitivity for detection of tumors in the urogenital system.

### TECHNICAL FIELD

The present invention pertains to the fields of genomics and bioinformatics, and relates to a classification method, device and use of urine sediment genomic DNA.

### BACKGROUND

Urogenital tumors refer to tumors that occur in the urinary system. Common urogenital tumors include renal cancer (RC), bladder tumor (BT) and prostate cancer (PCA). The Cancer Statistics Report in 2018 shows that, among the top 20 common tumors in terms of new cases and death cases, there are three urogenital tumors and PCA is in top three.

Most of the patients with early-stage tumors can be radically cured by surgeries, but the prognosis and survival of patients are significantly reduced once metastases occur. Currently, the diagnosis of urogenital tumors mainly relies on tissue biopsies, while non-invasive diagnosis is immature, and the sensitivity and specificity in tumor detection are not high.

Renal cell carcinoma is also known as renal cancer, and a common subtype is kidney renal clear cell carcinoma, accounting for about 80-85% of renal cancer. The main types of renal cancer include kidney renal clear cell carcinoma, papillary renal cell carcinoma, and chromophobe renal cell carcinoma, which together account for about 95% of renal cancer. Due to lack of good markers for early diagnosis, renal cell carcinoma has progressed to advanced stages at the time of diagnosis in many patients.

Currently, the clinically recognized “gold standard” for the diagnosis and follow-up of BT relies on the combination of cystoscopy with pathological examination on shed cells in urine. The entire bladder can be examined by cystoscopy, but cystoscopy has a low diagnostic sensitivity (52%-68%) for high-grade bladder carcinoma in situ. In addition, the friction of the instrument against the urethra during the examination can easily lead to urothelial injury to a patient, resulting in a strong sense of pain to the patient. The diagnostic sensitivity of pathological examination on shed cells in urine is low, especially for BT with low pathological grade (4%-31%).

Prostate specific antibody (PSA) tests are widely used in the process of early diagnosis of prostate cancer. However, the PSA variation is susceptible to many factors, making its accuracy not high. Furthermore, prior to paracentesis, the selective use of multi-parameter parametric magnetic imaging (mpMRI) may improve the detection rate of prostate cancer (Gleason score >7). However, the use of mpMRI is controversial, and further diagnosis must rely on pathological diagnosis.

Liquid biopsy refers to a technique for detecting dynamic changes in tumors by using circulating tumor cells (CTCs), cell-free tumor DNAs, and exosomes released by tumor tissue into body fluids such as blood and urine. Due to its non-invasive or minimally invasive, real-time and dynamic characteristics, liquid biopsy has been widely used in the research of early diagnosis, metastasis, prognosis judgment, mechanisms of forming drug resistance and personalized treatment guidance of tumors. Currently, most of the studies on liquid biopsy mainly use blood as a carrier. In fact, the advantage of urine over blood is pronounced, i.e. truly non-invasive.

However, similar to liquid biopsy which uses blood as a carrier, urine-based liquid biopsy technology faces the problem of how to make use of a limited signal to trace the origin of a tumor tissue due to the low level of signal released by urogenital tumors. Currently, genomic variation tracing based on NGS technology has been reported, including driver gene mutations, and insertions and deletions. However, tumors are highly heterogeneous, and the driver gene variation may not be detected in shed cells. Furthermore, the identification of a mutation in a small number of tumor cfDNAs relies on targeted deep sequencing (>5000*) which may have sequencing errors.

At present, there is still a need to develop new means having good specificity and sensitivity for the detection of urogenital tumors. Such means is more convenient for multiple, long-term and prognostic monitoring, and reduces the suffering of patients.

### SUMMARY OF THE INVENTION

With comprehensive research and efforts, the inventors of the present application developed, for the first time, a method of screening classification markers by detecting copy number variations (CNVs) and methylation haplotype load (MHL) of DNA methylation haplotype blocks (MHBs) in urine sediment genomic DNAs, and further developed a method of diagnosing urogenital tumors with high sensitivity and specificity, which can not only well distinguish tumor patients from healthy people, but also localize urogenital tumors. In addition, a prognostic survival model and corresponding 9 bladder cancer prognostic markers and 16 renal cancer prognostic markers were constructed by integrating clinical prognostic data from bladder cancer and renal cancer. Therefore, the following inventions are provided.

One aspect of the present application relates to a DNA classification method, comprising

calculating the MHL value or β mean of a DNA methylation haplotype block of a sample of interest and/or calculating the DNA copy number variation data of the sample of interest; and

calculating the similarity between the MHL value or β mean of the DNA methylation haplotype block of the sample of interest DNA and the MHL value or β mean of a DNA methylation haplotype block of a respective classification label, and/or calculating the similarity between the DNA copy number variation data of the sample of interest and the DNA copy number variation data of the respective classification label; and

determining the classification for the DNA in the sample of interest by using a classifier model and based on the similarity.

Preferably, the β mean is obtained by 450K chip data or 850K chip data.

In one or more embodiments of the present application, in the DNA classification method, the MHL value of the DNA methylation haplotype block and the DNA copy number variation data of a sample of interest are calculated; and the similarity between the MHL value of the DNA methylation haplotype block of the sample of interest and the MHL value of the DNA methylation haplotype block of a respective classification label, and the similarity between the DNA copy number variation data of the sample of interest and the DNA copy number variation data of a respective classification label are calculated.

In one or more embodiments of the present application, in the DNA classification method, the MHL value of the DNA methylation haplotype block of a sample of interest is calculated; and the similarity between the MHL value of the DNA methylation haplotype block of the sample of interest and the MHL value of the DNA methylation haplotype block of a respective classification label is calculated.

In one or more embodiments of the present application, in the DNA classification method, a β mean of a DNA methylation haplotype block of a sample of interest is calculated; and the similarity between the β mean of the DNA methylation haplotype block of the sample of interest and the β mean of the DNA methylation haplotype block of a respective classification label is calculated.

In one or more embodiments of the present application, in the DNA classification method, determining the classification for the DNA in the sample of interest comprises

determining a correlation between the MHL value of the DNA methylation haplotype block of a respective classification label and a human urogenital tumor, and/or a correlation between the DNA copy number variation data of a respective classification label and a human urogenital tumor by using a random forest model and based on the similarity; and

determining the classification for the DNA in the sample of interest by using the classifier model and based on the correlation.

In one or more embodiments of the present application, in the DNA classification method, determining the correlation between the MHL value of the DNA methylation haplotype block of a respective classification label and a human urogenital tumor comprises, based on the correlation, ranking the MHL value of the DNA methylation haplotype block to form a vector sequence, and inputting the vector sequence into the random forest model to determine a correlation between the MHL value of the DNA methylation haplotype block and a human urogenital tumor;

and/or

determining the correlation between the DNA copy number variation data of a respective classification label and a human urogenital tumor comprises, based on the correlation, ranking the DNA copy number variation data to form a vector sequence, and inputting the vector sequence into the random forest model to determine a correlation between the DNA copy number variation data of the classification label and a human urogenital tumor.

In one or more embodiments of the present application, in the DNA classification method, the human urogenital tumor is any one, any two (prostate cancer and urothelial cancer, urothelial cancer and renal cancer, or prostate cancer and renal cancer), or all three selected from the group consisting of prostate cancer, urothelial cancer, and renal cancer;

preferably, the renal cancer is a kidney renal clear cell carcinoma,

preferably, the urothelial cancer is upper tract urothelial cancer and/or bladder cancer,

preferably, the prostate cancer is prostate adenocarcinoma; and

preferably, the human urogenital tumor is diagnosed by biopsy from a surgery.

In one or more embodiments of the present application, in the DNA classification method, the random forest model includes at least three random forest binary classifiers and is selected from any one, any two, any three or all four of the following groups I-VI:

I. normal-vs-renal cancer, normal-vs-urothelial cancer, and normal-vs-prostate cancer;

II. renal cancer-vs-normal, renal cancer-vs-urothelial cancer, and renal cancer-vs-prostate cancer;

III. urothelial cancer-vs-normal, urothelial cancer-vs-renal cancer, and urothelial cancer-vs-prostate cancer; and

IV. prostate cancer-vs-normal, prostate cancer-vs-renal cancer, and prostate cancer-vs-urothelial cancer.

In one or more embodiments of the present application, the DNA classification method comprises voting for each group, and determining the group with the highest number of votes as the final classification, wherein if equal numbers of votes occur, the category with the highest prediction probability among the groups with the equal number of votes is determined as the final classification.

Since it is theoretically impossible for a female to be predicted to have prostate cancer, if a female sample is predicted to be prostate cancer, a sub-optimal prediction result is taken. For example, if the vote predicted to be renal cancer is second only to prostate cancer, the predictive label of the female sample is defined as renal cancer. If equal numbers of votes occur in groups, the probabilities in the groups are compared. The category with higher probability is determined as the final prediction result of the female sample.

In one or more embodiments of the present application, in the DNA classification method, the sample is a urine sample, preferably urina sanguinis, and more preferably, urine sediment of the urina sanguinis. Urine sediment can be obtained via technical means known to a person skilled in the art, for example, by centrifuging a urine sample and removing the supernatant; and preferably, the centrifugation is performed at a temperature less than or equal to 4° C.

In one or more embodiments of the present application, in the DNA classification method, the MHL value of the DNA methylation haplotype block of the sample of interest, the MHL value of the DNA methylation haplotype block of a respective classification label, the DNA copy number variation data of the sample of interest, and the DNA copy number variation data in a respective classification label are all calculated from the sequencing data of the DNAs in the urine sample;

preferably, the DNAs in the urine sample are urine sediment DNAs; and

preferably, the sequencing data is whole genome methylation sequencing data, such as whole genome bisulfite sequencing (WGBS) data; and preferably, the sequencing depth is 1×-5×.

In one or more embodiments of the present application, in the DNA classification method, the DNA methylation haplotype block of the sample of interest is the same as the DNA methylation haplotype block of a respective classification label; and/or

the DNA copy number variation regions of the sample of interest are the same as the DNA copy number variation regions of a respective classification label;

preferably, the methylation haplotype blocks and the copy number variation regions are those as shown in any one, any two, any three, any four, any five or all six of Tables 1-6, or as shown in Table 11 and/or Table 12.

In one or more embodiments of the present application, in the DNA classification method, the MHL value of the DNA methylation haplotype block of the sample of interest and the MHL value of DNA methylation haplotype block of a respective classification label are calculated by using MONOD2 software, and/or the DNA copy number variation data of the sample of interest and the DNA copy number variation data of a respective classification label are calculated by using Varbin;

preferably, the MHL value corresponding to the respective methylation haplotype block in the WGBS data is calculated by using MONOD2 software, and/or the copy number variation data corresponding to the respective copy number variation region in the WGBS data is calculated by using Varbin, wherein the methylation haplotype block and the copy number variation region are those as shown in any one, any two, any three, any four, any five, or all six of Table 1-6, or as shown in Table 11 and/or Table 12.

In one or more embodiments of the present application, in the DNA classification method, the DNA copy number variation data of the sample of interest and/or the DNA copy number variation data of a respective classification label are calculated in the following way.
Dividing the genome of a test sample into 5,000 to 500,000 bins of equal length or the same theoretical simulated copy number, normalizing the sequencing data, and calculating the ratio A/B of the number of reads corresponding to each bin, wherein:
A is the number of actual reads corrected for GC content in a bin;
B is the number of theoretical reads in the bin, which is obtained by dividing the total number of reads detected in the sample by the total number of bins; and
the ratio A/B is the copy number variation.
In one or more embodiments of the present application, in the DNA classification method, the genome of the test sample is divided into 5,000 to 500,000 bins of equal length or the same theoretical simulated copy number by Varbin, CNVnator, ReadDepth or SegSeq;
and/or
the ratio A/B of the number of reads corresponding to each bin is calculated by Varbin, CNVnator, ReadDepth or SegSeq.

In one or more embodiments of the present application, in the DNA classification method, the biomarker is a DNA segment from a start position S±m to a termination position T±n on a chromosome;

wherein S is a start site, T is a termination site, and the start and termination sites are those as shown in any one, any two, any three, any four, any five, or all six of Tables 1-6, or the start and termination sites are those as shown in Table 11 and/or Table 12; and

wherein m and n are independently non-negative integers less than or equal to 6000.

In one or more embodiments of the present application, in the DNA classification method, m and n are independently 5000, 4000, 3000, 2000, 1500, 1000, 500, 300, 200, 150, 100, 90, 80, 70, 60, 50, 40, 30, 20, 10, 5, or 0.

Another aspect of the present application relates to a method for the detection, diagnosis, classification, risk assessment or prognostic assessment of a human urogenital tumor, comprising

(1) obtaining a urine sample and extracting urine sediment DNAs;

(2) fragmenting the DNAs into fragments of 300-500 bp;

(3) constructing a whole genome library, preferably a whole genome methylation sequencing library, such as a whole genome bisulfite sequencing library, using the obtained DNA fragments; and

(4) classifying the DNA fragments in the library using any DNA classification method described in the present application, wherein the DNA fragments serve as the DNA in the sample of interest.

In one or more embodiments of the present application, in the method for the detection, diagnosis, classification, risk assessment, or prognostic assessment of a human urogenital tumor, the urogenital tumor is one or more selected from the group consisting of prostate cancer, urothelial cancer, and renal cancer; and preferably, the renal cancer is kidney renal clear cell carcinoma, the urothelial cancer includes upper tract urothelial cancer and bladder cancer, and the prostate cancer is prostate adenocarcinoma.

In one or more embodiments of the present application, in the method for the detection, diagnosis, classification, risk assessment or prognostic assessment of a human urogenital tumor, in step (1), the urine sample is urina sanguinis; and preferably, the urine sample is urine sediment of the urina sanguinis.

In one or more embodiments of the present application, in the method for the detection, diagnosis, classification, risk assessment or prognostic assessment of a human urogenital tumor, in step (2), the DNAs are fragmented into fragments of 350-450 bp.

A further aspect of the present application relates to a device for the detection, diagnosis, classification, risk assessment or prognostic assessment of a human urogenital tumor, comprising:

I. ‘normal decision unit’:

normal-vs-renal cancer, normal-vs-urothelial cancer, and normal-vs-prostate cancer;

II. ‘renal cancer decision unit’:

renal cancer-vs-normal, renal cancer-vs-urothelial cancer, and renal cancer-vs-prostate cancer;

III. ‘urothelial cancer decision unit’:

urothelial cancer-vs-normal, urothelial cancer-vs-renal cancer, and urothelial cancer-vs-prostate cancer;

IV. ‘prostate cancer decision unit’:

prostate cancer-vs-normal, prostate cancer-vs-renal cancer, and prostate cancer-vs-urothelial cancer,

preferably, the decision units can perform any DNA classification method described in the present application.

A further aspect of the present application relates to a device for the detection, diagnosis, classification, risk assessment or prognostic assessment of a human urogenital tumor, comprising

a memory; and

a processor coupled to the memory;

wherein program instructions which can be executed by the processor are stored in the memory, and the program instructions include any one, any two, any three, or all four decision units selected from the group consisting of

I. ‘normal decision unit’:

normal-vs-renal cancer, normal-vs-urothelial cancer, and normal-vs-prostate cancer;

II. ‘renal cancer decision unit’:

renal cancer-vs-normal, renal cancer-vs-urothelial cancer, and renal cancer-vs-prostate cancer;

III. ‘urothelial cancer decision unit’:

urothelial cancer-vs-normal, urothelial cancer-vs-renal cancer, and urothelial cancer-vs-prostate cancer;

IV. ‘prostate cancer decision unit’:

prostate cancer-vs-normal, prostate cancer-vs-renal cancer, and prostate cancer-vs-urothelial cancer;

wherein each decision unit comprises three random forest binary classifiers.

In one or more embodiments of the present application, for the device, the processor is configured to perform any classification method described in the present application based on the instructions stored in the memory.

In one or more embodiments of the present application, for the device, the urogenital tumor is one or more selected from the group consisting of prostate cancer, urothelial cancer, and renal cancer;

preferably, the renal cancer is a kidney renal clear cell carcinoma,

preferably, the urothelial cancer is upper tract urothelial cancer and/or bladder cancer, and

preferably, the prostate cancer is prostate adenocarcinoma.

A further aspect of the present application relates to the use of any one of the following items 1) to 3) in the preparation of a medicament for the detection, diagnosis, risk assessment or prognosis assessment of a human urogenital tumor:

1) the biomarkers described in the present application (i.e., the methylation haplotype blocks and/or the copy number variation regions);

2) DNAs in human urine, in particular in the urine sediment of human urine;

preferably, the urine is urina sanguinis, and

preferably, the DNAs are 300-500 bp, such as 350-450 bp, in length;

3) A DNA library prepared from item 2); preferably, the DNA library is a whole genome library, preferably a whole genome methylated sequencing library such as a whole genome bisulfite sequencing library;

preferably, the urogenital tumor is one or more selected from the group consisting of prostate cancer, urothelial cancer, and renal cancer;

preferably, the renal cancer is a kidney renal clear cell carcinoma,

preferably, the urothelial cancer is upper tract urothelial cancer and/or bladder cancer, and

preferably, the prostate cancer is prostate adenocarcinoma.

The present application also relates to a set of biomarkers (i.e., the methylation haplotype blocks and/or the copy number variation regions), wherein a biomarker is a DNA segment from a start position S±m to a termination position T±n on a chromosome;

wherein S is a start site, T is a termination site, and the start and termination sites are those as shown in any one, any two, any three, any four, any five, or all six of Tables 1-6, or the start and termination sites are those as shown in Table 11 and/or Table 12; and

wherein m and n are independently non-negative integers less than or equal to 6000.

In one or more embodiments of the present application, for the biomarkers, m and n are independently 5000, 4000, 3000, 2000, 1500, 1000, 500, 300, 200, 150, 100, 90, 80, 70, 60, 50, 40, 30, 20, 10, 5, or 0.

Some terms involved in the present application are explained below.

The term “bin” (section/region) is a generic description about artificially defining or dividing a genome by a certain length in the field of genomics. For example, if the human genome of about 3 billion base pairs is divided into 3000 bins on average, the size of each bin is about one million base pairs.

The term “coverage” refers to the proportion of a region of the genome that has been detected at least once accounting for the entire genome. Coverage is a term used to measure the extent to which the genome is covered by data. Due to the presence of complex structures (such as high GC and repeat sequences) in the genome, the final sequence obtained by sequencing, splicing and assembling often cannot cover all regions, and the regions which cannot be obtained are referred to as Gap. For example, when a bacterial genome is sequenced, and the coverage is 98%, 2% of the sequence region is not obtained by sequencing.

The term “sequencing depth” refers to the ratio of the total number of bases (bp) obtained by sequencing to the size of the genome, or it is understood as the average number of times that each base in the genome is sequenced. For example, assuming that the size of a gene is 2M and the obtained total amount of data is 20M, the sequencing depth is 20M/2M=10×.

The term “reads” or “read” refers to a read fragment, i.e., a read sequence.

The term “pair-end reads” refers to paired reads.

The term “copy number variations (CNVs)” refers to a deletion or duplication of a relatively large DNA fragment, typically an increase or a decrease in the copy number of DNA fragments of hundreds of bp to millions of bp. CNVs are caused by genomic rearrangements and are one of the important pathogenic factors of tumors. In one embodiment of the present application, the copy number variation is calculated in the following way.

The genome of a test sample is divided into 5,000-500,000 bins (e.g., 50,000 bins) of equal length or the same theoretical simulated copy number. The ratio A/B of the read number corresponding to each bin is calculated by software or algorithms such as Varbin, CNVnator, ReadDepth or SegSeq (A is the number of actual reads corrected for the GC content in a bin; B is the number of theoretical reads in the bin, which is obtained by dividing the total number of reads read in the sample by the total number of bins). The ratio A/B is the copy number variation.

The term “theoretical simulated copy number” involves dividing a genome into several regions of equal or unequal length by a software and/or method of calculating copy number, where theoretical copy number contained in each region is same by data simulation.

The term “MHB” refers to DNA methylation haplotype blocks, also referred to herein as DNA methylation haplotype region or DNA methylation haplotype modules, meaning a linkage region in which DNA co-methylation frequently occurs in the genome. The basic principle is based on the co-methylation linkage of adjacent CpG sites. The algorithm extends the concept of linkage disequilibrium (LD) in traditional genetics, which indicates the degree of co-methylation of adjacent CpG sites in DNA methylation, that is, the linkage condition of DNA methylation. The linkage condition of adjacent CpG sites is first calculated by DNA methylation haplotype, and the region with r² not less than 0.5 in adjacent CpG sites is further defined as potential MHBs. The potential MHBs are then expanded according to the overlapping CpG sites in the MHB region, and final MHBs are obtained. They can be identified by using technical means known to a person skilled in the art, for example, by using MONOD2 software (http://genome-tech.ucsd.edu/public/MONOD_NG_TR44413/scripts_and_codes/) developed by Kun Zhang's Research Team.

The term “MHL” refers to DNA methylation haplotype load, which represents the heterogeneous distribution of different DNA methylation haplotypes in a given region, i.e., the proportion of CpG site methylation modifications.

The term “TNM” represents a tumor staging system in which:

“T” is the initial letter of the wording “tumor”, and refers to the size or direct extent of a primary tumor. With an increase in tumor volume and an increase in the extent of adjacent tissue involvement, it is represented by T1˜T4 in turn.

“N” is the initial letter of the wording “Node”, and refers to the involvement of regional lymph node. When the lymph node is not involved, it is represented by N0. With an increase of the degree and extent of lymph node involvement, it is represented by N1˜N3 in turn.

“M” is the initial letter of the wording “metastasis” and refers to distant metastasis (usually hematogenous metastasis). No distant metastasis is represented by M0 and the presence of distant metastasis is represented by M1. On this basis, a specific stage is delineated by the grouping of the three indicators of TNM.

### Advantageous Effects

One or more of the following technical effects are achieved in the present application.

(1) Non-invasive diagnosis in the true sense. Sampling is simple, which only requires obtaining a certain volume of urina sanguinis, and there is no trauma to the subjects. This is advantageous for sample collection, diagnosis, long-term monitoring and regular monitoring of prognosis.

(2) High success rate of library construction. The amount of urine sediment DNAs is much more than that of urine cell-free DNAs, so that the amount of starting DNAs for library construction is much more than that of cfDNAs for library construction. In addition, there are kits available for library construction and sequencing, which makes the operation easier and more stable and reliable.

(3) Low-depth high-throughput sequencing. In the present application, the integration of the information of DNA methylation and DNA copy number variation and the extraction of a tumor signal in a unit of a region by optimizing a modeling algorithm can not only maximumly retain the tumor signal, but also maximumly reduce sequencing cost. Theoretically, it is possible to obtain a result with high sensitivity and specificity at a sequencing depth of about 1× to 5×.

(4) High-accuracy diagnosis of a single tumor. The diagnosis and recurrence monitoring of common tumors of the urinary system (such as renal cancer, bladder cancer and prostate cancer) can be achieved using the constructed binary classifier model.

(5) Tumor localization. The use of the multi-stage classification system of the present application can not only determine whether a tumor is present or not, but also locate the potential tumor type of a tumor patient.

(6) Potential application in prognostic risk assessment. The prognostic markers screened by the present application can be potentially applied to the survival prognostic assay in a tumor patient.

### BRIEF DESCRIPTION OF THE DRAWINGS

FIG. 1. Flow chart for data generation and analysis of models for non-invasive diagnosis, localization, and prognosis of urogenital tumors. The DNA methylation haplotype blocks (MHBs), copy number variations (CNVs), and DNA methylation profile of urine sediment are identified by low-depth whole-genome bisulfite sequencing (SWGBS). CNVs and/or MHB markers in urine sediment (cancer patients vs. healthy people) and tumor tissues (tumor tissues vs. pericarcinomatous tissues) are selected by random forest machine learning algorithm for further feature selection. These features are then used to construct a binary classifier, a multivariate classifier, and a prediction model. These models have potential applications in the diagnosis, localization and prognosis of urogenital tumors.

FIG. 2A. Schematic diagram of feature selection of urothelial cancer. Random forest algorithm is used for the feature selection. FN: number of features. The number of features in the model is determined by the accuracy and kappa coefficient. Feature filtering is based on the importance weight of a feature in the model. In the TCGA methylation 450K data (F1) and the WGBS data (F2), the feature selection requires not only a methylation difference between a tumor tissue and a normal tissue, but also a DNA methylation difference between urine sediment of a tumor patient and a healthy person. The union of F1 and F2 and further filtering results are defined as F3. Similarly, the feature selection of CNVs of urine sediment also requires that the feature can distinguish not only a normal tissue from a cancer tissue, but also a healthy person and a tumor patient, and the result is defined as f4. The features of DNA methylation f3 and copy number variations (CNVs) f4 are integrated, and further screening results are defined as f5.

FIG. 2B. Comparison of methylation haplotype load (MHL) with four other methods for calculating methylation haplotypes. Five pattern combinations of methylation haplotypes (schematics) are used to illustrate methylation frequency, DNA methylation entropy, Epi-polymorphism, methylation haplotypes, and MHL. MHL is the only indicator that can distinguish all five patterns.

FIG. 2C. Schematic representation of a selection of urothelial cancer vs. healthy F1. The number of features in the model is determined by the accuracy and kappa coefficient of the model training process. When model performance is optimal, the black arrow points to the number of selected features.

FIG. 2D. Schematic representation of a selection of renal cancer vs. healthy F1. The number of features in the model is determined by the accuracy and kappa coefficient of the model training process. When model performance is optimal, the black arrow points to the number of selected features.

FIG. 2E. Schematic representation of a selection of prostate cancer vs. healthy F1. The number of features in the model is determined by the accuracy and kappa coefficient of the model training process. When model performance is optimal, the black arrow points to the number of selected features.

FIG. 2F. ROC graph of validating F1 and F4, which is screened by the constructed binary classifier of urothelial cancer vs. healthy, in the TCGA bladder cancer dataset. AUC represents the area under the curve. The solid line ROC graph represents the result of validating F1 in TCGA. The dashed ROC graph represents the result of validating F4 in TCGA.

FIG. 2G. ROC graph of validating F1 and F4, which is screened by the constructed binary classifier of renal cancer vs. healthy, in the TCGA renal cancer dataset. AUC represents the area under the curve. The solid line ROC graph represents the result of validating F1 in TCGA. The dashed ROC graph represents the result of validating F4 in TCGA.

FIG. 2H. ROC graph of validating F1 and F4, which is screened by the constructed binary classifier of prostate cancer vs. healthy, in the TCGA prostate cancer dataset. AUC represents the area under the curve. The solid line ROC graph represents the result of validating F1 in TCGA. The dashed ROC graph represents the result of validating F4 in TCGA.

FIG. 3A. Flow chart of the construction of GUseek (a multi-stage classifier) consisting of four decision systems, each of which consists of three binary classifiers. For an unknown type of sample, it is first assigned to four decision systems for prediction and the corresponding scores and probabilities of prediction categories are obtained. Next, the unknown sample is labeled by comparing the scores of different prediction categories. The prediction category with the highest score is the prediction result of GUSeek (a multi-stage classifier). The prediction categories with the same score are further compared with their prediction probabilities. The category with the highest probability is taken as the final prediction category.

FIG. 3B. Comparison of GUseek with six other multi-class classification machine learning algorithms in 10 times of random modeling and the average overall accuracy of the corresponding predictions. RF: Random Forest, SVM: Support Vector Machine, LDA: Linear Discriminant Analysis, LASSO: Lasso Algorithm, KNN: k-Nearest Neighbor, and Bayes: Bayesian Algorithm.

FIG. 4A. Flow chart of constructing a prognostic model using markers of DNA methylation and urine sediment CNVs.

FIG. 4B. ROC graph of a prognosis model for bladder cancer. The black solid line is a prognostic model that integrates DNA methylation with clinical features, the gray solid line is a prognostic model constructed with only clinical features, the dashed line is a prognostic model constructed with only DNA methylation information, and the corresponding area under the curve (AUC) decreases in turn.

FIG. 4C. ROC graph of a prognosis model for renal cancer. The black solid line is a prognostic model that integrates DNA methylation and clinical features, the dashed line is a prognostic model constructed with only DNA methylation information, the gray solid line is a prognostic model constructed with only clinical features, and the corresponding area under the curve (AUC) decreases in turn.

FIG. 4D. K-M survival curve corresponding to all datasets of bladder cancer. There are significant differences between a high-risk group and a low-risk group.

FIG. 4E. K-M survival curve corresponding to a training set of bladder cancer. There are significant differences between a high-risk group and a low-risk group.

FIG. 4F. K-M survival curve corresponding to a test set of bladder cancer. There are significant differences between a high-risk group and a low-risk group.

FIG. 4G. K-M survival curve corresponding to all datasets of renal cancer. There are significant differences between a high-risk group and a low-risk group.

FIG. 4H. K-M survival curve corresponding to a training set of renal cancer. There are significant differences between a high-risk group and a low-risk group.

FIG. 4I. K-M survival curve corresponding to a test set of renal cancer. There are significant differences between a high-risk group and a low-risk group.

### DETAILED DESCRIPTION

The embodiments of the present application will be described in detail below in reference to Examples. It should be understood by a person skilled in the art that the following Examples are merely illustrative of the present application and are not intended to limit the scope of the present application. The experimental methods without specifying their protocols in the Examples are generally carried out according to conventional protocols, or according to protocols recommended by manufacturers. The used reagents or the instruments without specifying the manufacturer are commercially available conventional products.

In the present application,

The 450K chip data refers to the Illumina Infiium Human Methylation 450 BeadChip chip technology developed by Illumina, where 450K refers to the number of probes on the chip, which can detect the corresponding number of methylation sites.

The 850K chip data refers to the Illumina Infiium Human Methylation 850 BeadChip chip technology developed by Illumina, where 850K refers to the number of probes on the chip, which can detect the corresponding number of methylation sites.

The TCGA snp6.0 chip data is provided by a public database, which can be downloaded, for example, from http://firebrowse.org/?cohort=PRA or https://portal.gdc.cancer.gov/. The number of copy number variations in the area covered by the SNP6.0 chip can be detected.

The available clinical data of the TCGA is provided by a platform for tumor research, which is provided by the TCGA official website (https://www.cancer.gov/). A person skilled in the art can also obtain the available clinical data of the TCGA by other integration software and online platforms, such as http://firebrowse.org/and software such as TCGA download widgets.

### Example 1: Preparation of DNA Samples

1. Subject Population

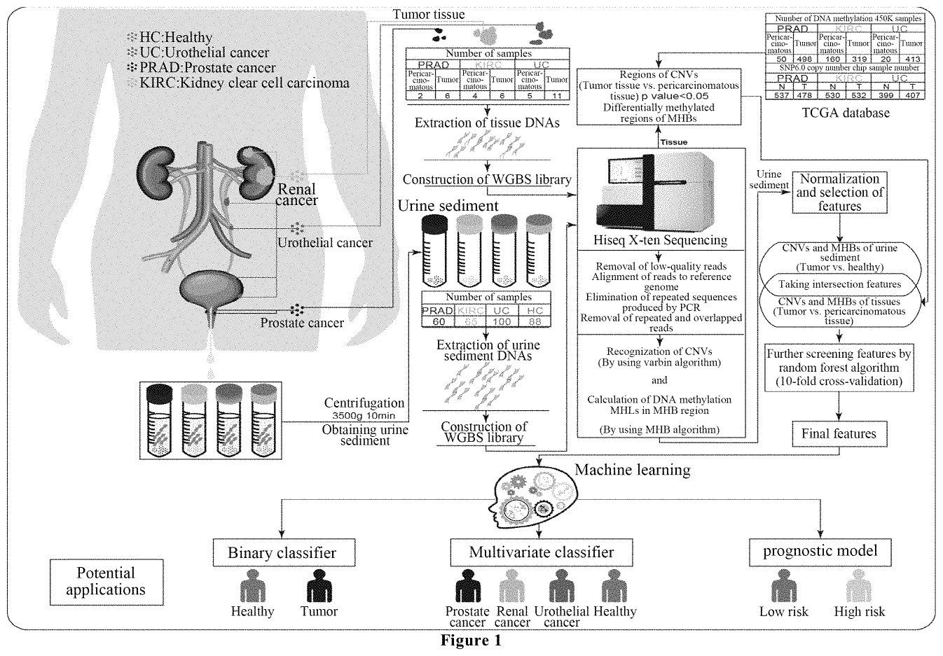

Urine samples from a total of 313 subjects were collected, as shown in FIG. 1. The 313 subjects included 88 healthy people (healthy), 65 patients with kidney renal clear cell carcinoma (KIRC), 100 patients with urothelial cancer (UC, including urinary bladder cancer (UBC), and upper tract urothelial cancer (UTUC)), and 60 patients with prostate cancer (PRAD).

2. Experimental Methods

(1) Fresh urine (urina sanguinis) from preoperative tumor patients and fresh urine (urina sanguinis) from healthy people were collected. The urines were collected in 50 ml centrifuge tubes with a volume of about 45-50 ml per urine sample.

(2) The collected urina sanguinis samples were centrifuged at 3500 rpm and 4° C. for 10 min, respectively. The supernatants were removed to obtain urine sediments.

(3) The urine sediments were washed twice with PBS buffer (500 ml of PBS buffer was added each time, and after centrifugation at 13000 g for 1 min, the supernatants were removed), and then the urine sediments were transferred to 1.5 ml EP tubes.

(3) Urine sediment genomic DNAs (urine sediment gDNAs) were extracted by using QIAamp DNA Mini Kit. After extraction, the concentration of the DNAs was measured with Qubit and the DNAs were stored at −80° C. for later use.

313 DNA samples were prepared.

### Example 2: Construction of a Whole Genome Bisulfite Sequencing (Abbreviated as BS-Sea or WGBS) Library

50-200 ng of the DNA samples obtained in Example 1 were taken, respectively, as the start DNAs for library construction and lambda DNAs (all CpG sites included unmethylated C) and 5 mC DNAs (all CpG sites included methylated C) were added in a ratio of 3:1000. The DNAs were then fragmented with a Covaris sonicator such that the major length peaks of the fragments were in a range of 400 bp. The fragmented DNAs were then end repaired with NEBNext Ultra II End Repair/dA-Tailing Module 96 rxns (Cat. No. E7546) and were polyadenylated (polyA). Then, methylation PE linkers were added by using NEBNext Ultra II Ligation Module, 96 rxns unit (Cat. No. E7595L).

The resulting water-soluble DNAs with linkers ligated (i.e., the library) were subjected to a bisulfite treatment by using a EZ DNA methyhlation Gold kit (Zymo Research). The specific procedures were performed in accordance with the instructions for use of the kit. Afterwards, the DNAs were purified, amplified by PCR, and the concentration of the DNAs was determined by using the nucleic acid and protein quantitative analyzer Qubit2.0 of Life Tech, obtaining a DNA library.

The resulting DNA library was sent to Novogene for quality control of library fragmentation and concentration using Agilent 2100 and AB17500 Fluorescent quantitative PCR instruments, respectively. There was no problem in library examination, thereby obtaining a BS-seq library of 313 urine sediment gDNA samples for subsequent library sequencing.

### Example 3: Sequencing by HiSeq X10 System

1. Test Samples:

The BS-seq library of 313 urine sediment gDNAs prepared in the above Example 2.

2. Experimental Methods

Novogene sequencing company was entrusted to perform whole-genome sequencing on the BS-seq library of 313 urine sediment gDNAs.

3. Experimental Results

The data (i.e., a fastq raw file) on 150 bp pair-end reads of the BS-seq library of 313 urine sediment gDNAs was obtained for subsequent data preprocessing and tumor marker analysis.

### Example 4: Pretreatment of Sequencing Data

The reads of the BS-seq library of 313 urine sediment gDNAs obtained by sequencing in Example 3 was first subjected to quality control by Trimmomatic (version: Trimmomatic-0.32), including removal of low-quality reads and linkers. Next, genomic alignment was performed using Bismark (version: bismark v0.14.5) alignment software and PCR repeat amplification reads (deduplication) were removed. Then, the overlap regions between reads were then removed using bamUtil (version: bamUtil_1.0.12) software. The resulting bam file was then used as a starting file for an analysis of DNA copy number and methylation. Finally, the output data coverage of each sample in the BS-seq library of 313 urine sediment gDNAs was approximately 1×-5×.

### Example 5: Screening and Validation of DNA Methylation Tumor Markers

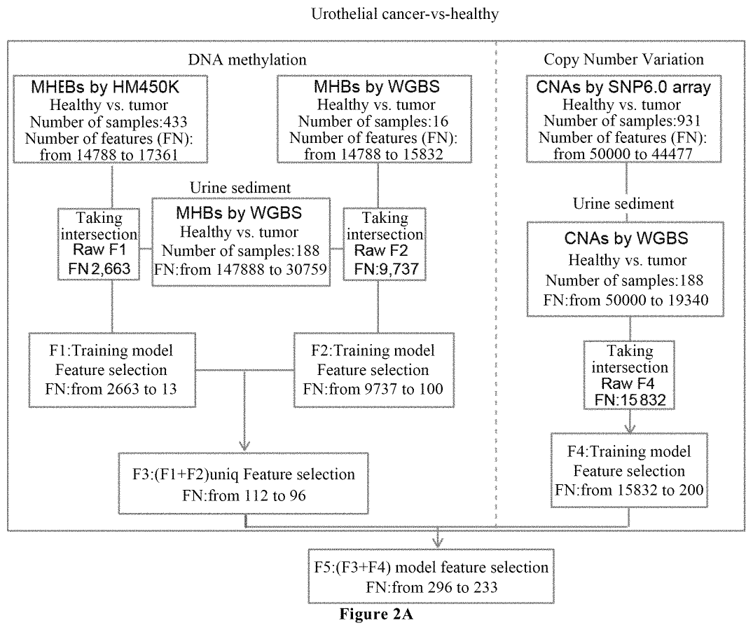

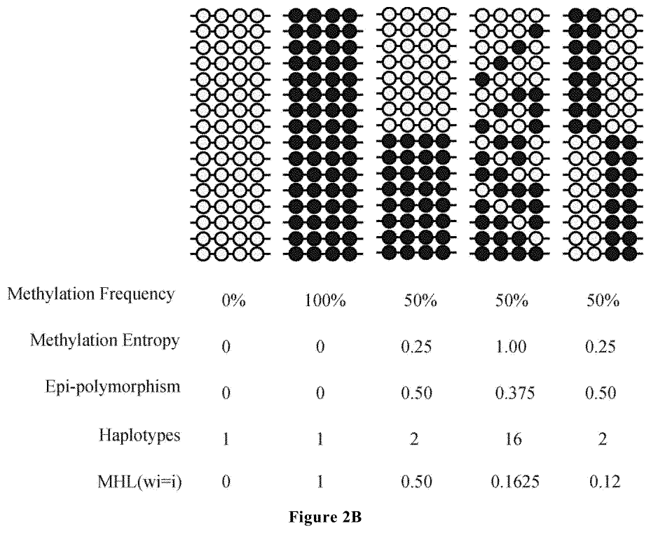

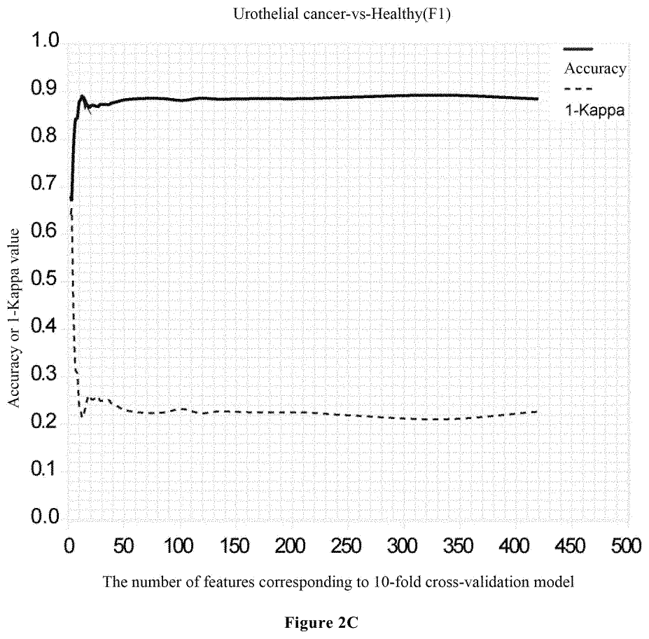

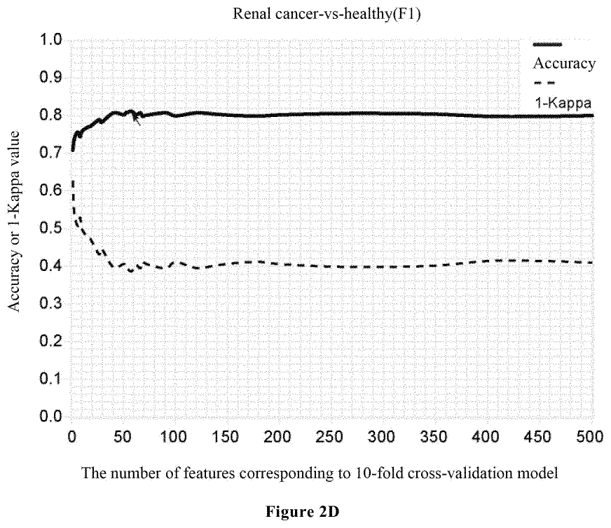

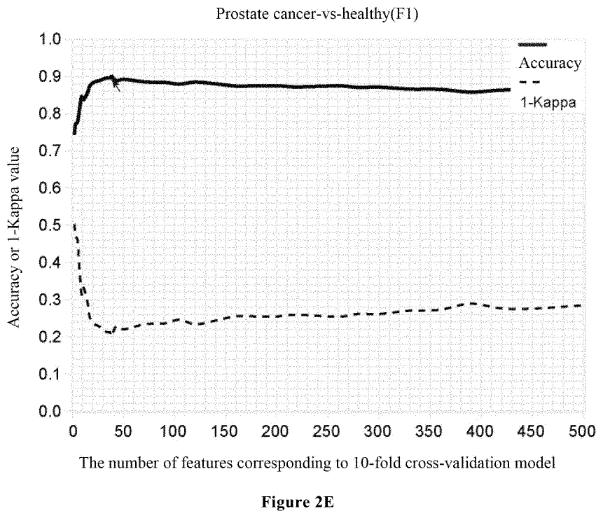

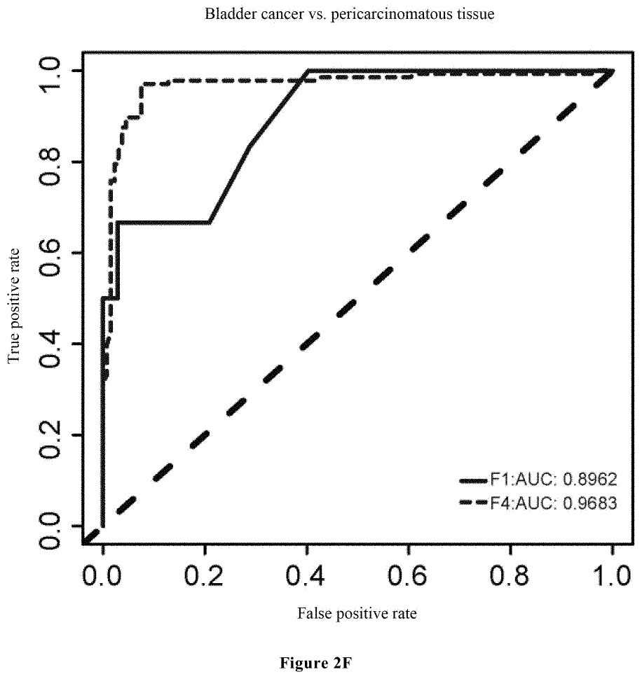

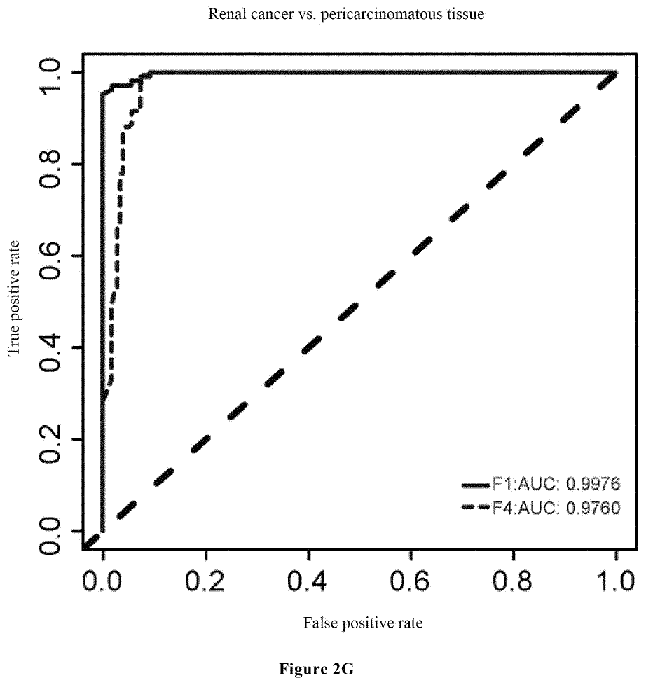

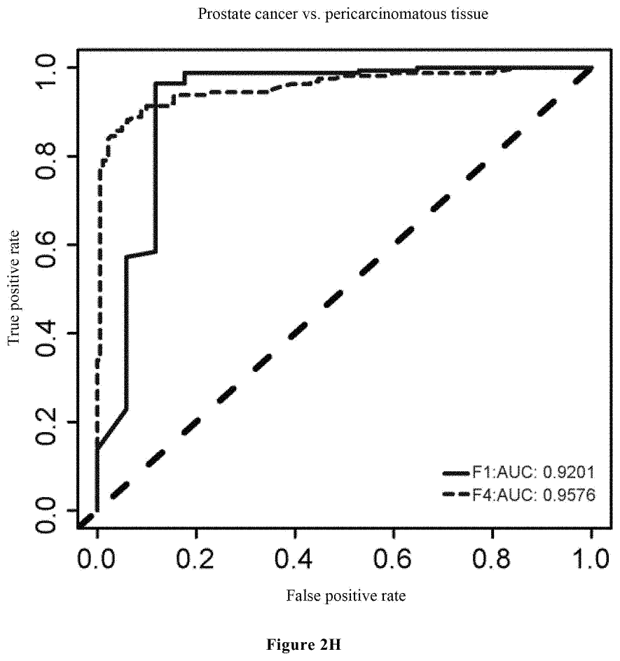

For the DNA methylation feature selection (shown in FIG. 2A), the inventors first utilized the published 147888 DNA methylation haplotype blocks (abbreviated as MHBs) in normal tissues (see Guo S, Diep D, Plongthongkum N, Fung H L, Zhang K, Zhang K. Identification of methylation haplotype blocks aids in deconvolution of heterogeneous tissue samples and tumor tissue-of-origin mapping from plasma DNA. Nature genetics. 2017; 49:635-42) as initial candidate features to calculate (calculation was performed according to the above analysis procedure with reference to the following website: http://genome-tech.ucsd.edu/public/MONOD_NG_TR44413/,) the value of methylation haplotype loads (abbreviated as MHL) of MHBs in 313 urine sediment samples. MHL was chosen because of its higher sensitivity. It can be seen from FIG. 2B that the other four methods for calculating the regional methylation haplotypes are not as good as MHL calculation. The other four methods for calculating the regional methylation haplotypes were as follows.

(1) Calculation of Methylation Frequency (average methylation level): for a given region, if the number of reads covering the base C was defined as Nc and the number of reads covering the base T was defined as Nt, the methylation level of the region was Nc/(Nc+Nt).

Reference: Chen, K. et al. Loss of 5-hydroxymethylcytosine is linked to gene body hypermethylation in renal cancer. Cell Research. 26(1):103-118 (2016).

(2) Calculation of Methylation Entropy (ME):

$$M E = - \frac{1}{b} {\sum }_{i = 1}^{n} P ( {H}_{i} ) * {log}_{2} P ( {H}_{i} )$$

wherein b denotes the number of corresponding CpG in a given region, n denotes the number of methylation haplotypes in a given region, and P (Hi) denotes the probability of observing a methylation haplotype in a given region.

Reference: Xie, H. et al. Genome-wide quantitative assessment of variation in DNA methylation patterns. Nucleic Acids Res. 39, 4099-4108 (2011).

(3) Calculation of Epi-polymorphism:

$$ppoly = 1 - {\sum }_{i = 1}^{n} {P}_{i}^{2}$$

The probability of occurrence of methylation haplotype i for a given region was Pi, and the number of methylation haplotypes was n.

Reference: Landan, G. et al. Epigenetic polymorphism and the stochastic formation of differentially methylated regions in normal and cancerous tissues. Nat. Genet. 44, 1207-1214 (2012).

(4) Calculation of Methylation Haplotypes

For a given region, the methylation status of the corresponding CpG covering reads was the methylation haplotype.

Reference: Shoemaker, R., Deng, J., Wang, W. & Zhang, K. Allele-specific methylation is prevalent and is contributed by CpG-SNPs in the human genome. Genome Res. 20, 883-889 (2010).

Where an MHL value cannot be calculated for an MHB because the sequenced reads did not cover the MHB, the MHL value of the MHB was filled with the average MHL value of the sample itself. The average MHL value was calculated as follows.

For each sample, there were 147888 MHBs to calculate MHLs. The MHBs where MHLs cannot be calculated were NA, and the corresponding number was n(NA). The MHL values were calculated if the MHBs of the MHLs can be calculated. The corresponding number was 147888-n(NA). The sum of all MHLs of the corresponding MHBs for which MHL values can be calculated the was Sum, and the average MHL value for each sample was Sum/(147888-n(NA)).

Finally, almost 150,000 MHBs containing MHL values can be obtained for each sample. These MHBs were used as initial candidate features for DNA methylation analysis. In order to narrow the range of screening features, the inventors divided the features into two groups.

One group was candidate raw F1, representing that the MHL values of some MHBs were different for the urine sediment gDNAs not only between the tumor patients and healthy people (student t-test, p value<0.05) (the difference analysis can use statistical analysis languages such as limma R package, student t-test test, and filter features by limiting the p-value threshold; or statistical analysis software such as SPASS, SAS, Metalab or Origin; similarly hereinafter), but also between the solid tumor tissues and the corresponding pericarcinomatous tissues in the TCGA methylation 450 K data (student t-test, p value<0.05).

The other group was candidate raw F2, representing that the MHL values of some MHBs were different for the urine sediment gDNAs not only between the tumor patients and healthy people (student t-test, p value<0.05), but also between the solid tumor tissue and the corresponding pericarcinomatous tissue in the constructed Whole Genome Bisulfite Sequencing (WGBS) data (student t-test, p value<0.05).

Next, MHBs were gradually kicked out for raw F1 and raw F2, respectively, until the accuracy (obtained by 10-fold cross-validation) and the kappa coefficient (the kappa coefficient was used for consistency test, and can also be used to measure classification accuracy, which was calculated based on a hybrid matrix) of the corresponding random forest model no longer increased. At this time, the obtained MHBs corresponded to F1 and F2 (as shown in FIG. 2C), respectively. F1 and F2 were combined into a hybrid matrix according to sample ID, and the MIHBs were further kicked out until the accuracy and the Kappa coefficient of the model training no longer increased, and the MHBs were defined as F3. F3 represented the final feature for DNA methylation.

In order to verify the reliability of the feature selection, the verification was performed by the inventors in combination with the TCGA methylation 450 K data. The verification method was as follows.

Firstly, using the screened F1 features, a β mean value of the F1 feature region corresponding to each sample was preliminarily calculated based on the TCGA 450K data (for a given region, if the number of 450K probes was n, and the sum of β values of all probes in the corresponding region was Sum β, then the average β value of the corresponding region was Sum_β/n), and then a hybrid matrix was constructed. Next, the samples were divided into a training set and a test set according to a ratio of 2:1. Then, the training set was modeled by a random forest algorithm, and the test set was used to test the predictive sensitivity and specificity of the model. Finally, the predictive performance of the model was displayed by combining the ROC curve.

The results showed that the selected feature could well distinguish a cancerous tissue from the corresponding pericarcinomatous tissue (as shown in FIGS. 2F-2H), indicating the accuracy of the F1 features of the present application.

### Example 6: Screening and Validation of CNV Tumor Markers

For the screening of subsequent feature of CNVs (F4) (as shown in FIG. 2A), the Varbin algorithm (Timour Baslan, et al. 2012. Nature protocols) was used. That is, the genome (the BS-seq data from in the above Example 4) was first divided into 50,000 bins, and then the number of reads in each bin was calculated and normalized based on the size of the sequencing library and the GC content to obtain the theoretical ratio of each region with respect to the expected value. Finally, 50,000 ratios could be obtained for each sample. These bins served as the initial candidate features for CNVs. Then, following CNVs were retained. The urine sediment gDNAs are different not only between the tumor patients and healthy people (student t-test, p value<0.05), but also between the tumor tissues and the corresponding pericarcinomatous tissues (student t-test, p value<0.05). Next, by using the random forest algorithm and 10-fold cross-validation method, the candidate features were gradually kicked out until the accuracy and the kappa coefficient of the corresponding random forest model no longer increased, at which time the remaining features were used as F4.

Similar to the F1 feature validation in Example 5, the inventors verified the F4 features using TCGA snp6.0 chip data. The results showed that the F4 features could well distinguish cancerous tissues from corresponding pericarcinomatous tissues (as shown in FIGS. 2F, 2G and 2H).

### Example 7: Data Integration and Establishment and Validation of Binary Classification Model

In order to further improve the model performance, the F3 features and the F4 features were integrated with reference to the method in Example 6. The candidate features were gradually kicked out until the accuracy and the kappa value of the model prediction no longer increased, at which time the remaining features were used as F5, as shown in Tables 1 to 6 below, where the importance was a result of output with importance parameters after the model was built using randomForest R package.

TABLE 1
Urothelial Cancer-vs-Healthy

| | Starting | Termination | | |
|---|---|---|---|---|
| Chromosome | Site | Site | Importance | Type |
| chr1 | 203293432 | 203293556 | 0.24 | MHB |
| chr1 | 237205772 | 237205848 | 0.18 | MHB |
| chr1 | 2375238 | 2375368 | 0.28 | MHB |
| chr1 | 74591750 | 74591856 | 0.21 | MHB |
| chr1 | 8431104 | 8431290 | 0.37 | MHB |
| chr11 | 48077655 | 48077813 | 0.10 | MHB |
| chr12 | 88254216 | 88254280 | 0.10 | MHB |
| chr13 | 114518802 | 114518814 | 0.47 | MHB |
| chr13 | 73615586 | 73615695 | 0.16 | MHB |
| chr15 | 91103514 | 91103705 | 0.20 | MHB |
| chr16 | 83152854 | 83153023 | 0.23 | MHB |
| chr19 | 53038840 | 53039091 | 0.09 | MHB |
| chr19 | 53039433 | 53039496 | 0.34 | MHB |
| chr2 | 66666351 | 66666409 | 0.13 | MHB |
| chr2 | 66667886 | 66667913 | 0.08 | MHB |
| chr2 | 66673054 | 66673077 | 0.11 | MHB |
| chr20 | 50618683 | 50618811 | 0.53 | MHB |
| chr20 | 54580409 | 54580415 | 0.08 | MHB |
| chr21 | 15914402 | 15914475 | 0.24 | MHB |
| chr21 | 37546252 | 37546419 | 0.80 | MHB |
| chr3 | 11623911 | 11624030 | 0.13 | MHB |
| chr3 | 152190054 | 152190208 | 0.39 | MHB |
| chr3 | 43431335 | 43431392 | 0.33 | MHB |
| chr3 | 5231207 | 5231346 | 0.23 | MHB |
| chr6 | 32920518 | 32920735 | 0.25 | MHB |
| chr7 | 28892934 | 28892987 | 0.40 | MHB |
| chr7 | 3018215 | 3018237 | 0.30 | MHB |
| chr8 | 64513914 | 64513934 | 0.33 | MHB |
| chr1 | 156406407 | 156406599 | 0.27 | MHB |
| chr1 | 166459242 | 166459289 | 0.58 | MHB |
| chr1 | 243646464 | 243646494 | 0.37 | MHB |
| chr1 | 54738815 | 54738862 | 0.61 | MHB |
| chr10 | 17470980 | 17471078 | 0.83 | MHB |
| chr10 | 27587575 | 27587656 | 0.58 | MHB |
| chr11 | 65374453 | 65374490 | 0.23 | MHB |
| chr12 | 103358958 | 103359251 | 0.45 | MHB |
| chr12 | 12171530 | 12171639 | 0.22 | MHB |
| chr12 | 24202022 | 24202282 | 1.10 | MHB |
| chr13 | 114475074 | 114475265 | 2.54 | MHB |
| chr13 | 25085404 | 25085494 | 0.23 | MHB |
| chr13 | 46755705 | 46756047 | 1.01 | MHB |
| chr15 | 31776089 | 31776103 | 0.19 | MHB |
| chr15 | 91472787 | 91472863 | 0.63 | MHB |
| chr16 | 19305414 | 19305566 | 0.40 | MHB |
| chr16 | 82979430 | 82979596 | 3.89 | MHB |
| chr17 | 62774654 | 62774697 | 0.32 | MHB |
| chr17 | 62775170 | 62775188 | 0.22 | MHB |
| chr18 | 32440694 | 32440860 | 2.02 | MHB |
| chr18 | 66711929 | 66712082 | 0.68 | MHB |
| chr19 | 29284698 | 29284703 | 0.18 | MHB |
| chr19 | 3404713 | 3404805 | 0.16 | MHB |
| chr19 | 4089228 | 4089390 | 0.80 | MHB |
| chr19 | 55463117 | 55463149 | 0.64 | MHB |
| chr2 | 102187418 | 102187570 | 1.41 | MHB |
| chr2 | 188309968 | 188310077 | 0.68 | MHB |
| chr2 | 196401030 | 196401147 | 0.72 | MHB |
| chr2 | 206276427 | 206276503 | 0.25 | MHB |
| chr21 | 23191793 | 23192016 | 0.17 | MHB |
| chr21 | 38069150 | 38069189 | 0.11 | MHB |
| chr3 | 105448762 | 105448959 | 0.38 | MHB |
| chr3 | 130086216 | 130086287 | 1.45 | MHB |
| chr3 | 161978029 | 161978179 | 0.22 | MHB |
| chr3 | 20145859 | 20146109 | 0.65 | MHB |
| chr3 | 95438485 | 95438560 | 0.33 | MHB |
| chr4 | 1397376 | 1397392 | 0.44 | MHB |
| chr4 | 24018497 | 24018685 | 0.30 | MHB |
| chr4 | 30878936 | 30879128 | 0.49 | MHB |
| chr4 | 54975988 | 54976001 | 0.44 | MHB |
| chr5 | 61728652 | 61728744 | 0.57 | MHB |
| chr5 | 68538415 | 68538647 | 0.48 | MHB |
| chr5 | 96016643 | 96016680 | 0.33 | MHB |
| chr6 | 108440389 | 108440510 | 0.07 | MHB |
| chr6 | 20320098 | 20320141 | 0.23 | MHB |
| chr6 | 47198472 | 47198580 | 0.25 | MHB |
| chr6 | 51658406 | 51658629 | 0.23 | MHB |
| chr7 | 116232750 | 116232819 | 0.50 | MHB |
| chr7 | 28548889 | 28549081 | 0.95 | MHB |
| chr7 | 7298626 | 7298766 | 1.32 | MHB |
| chr8 | 14336069 | 14336222 | 0.46 | MHB |
| chr8 | 41121887 | 41122005 | 0.84 | MHB |
| chr9 | 114881474 | 114881621 | 1.14 | MHB |
| chr9 | 115517974 | 115518223 | 0.50 | MHB |
| chr9 | 76788347 | 76788510 | 0.73 | MHB |
| chr9 | 971674 | 971703 | 0.21 | MHB |
| chr1 | 27311241 | 27366267 | 0.26 | CNV |
| chr1 | 75153840 | 75208962 | 0.22 | CNV |
| chr1 | 188229077 | 188284311 | 0.18 | CNV |
| chr1 | 218478067 | 218533154 | 0.23 | CNV |
| chr2 | 18766910 | 18822632 | 0.44 | CNV |
| chr2 | 19864110 | 19919131 | 0.23 | CNV |
| chr2 | 137082138 | 137137160 | 0.13 | CNV |
| chr2 | 231561625 | 231616899 | 0.29 | CNV |
| chr2 | 232446700 | 232501721 | 0.36 | CNV |
| chr3 | 4147446 | 4204099 | 0.13 | CNV |
| chr3 | 5877424 | 5932438 | 0.37 | CNV |
| chr3 | 7995424 | 8050438 | 0.26 | CNV |
| chr3 | 8050438 | 8107141 | 0.19 | CNV |
| chr3 | 8273493 | 8328506 | 0.30 | CNV |
| chr3 | 8386028 | 8442539 | 0.09 | CNV |
| chr3 | 8894104 | 8949118 | 0.94 | CNV |
| chr3 | 14819960 | 14875310 | 0.18 | CNV |
| chr3 | 16326396 | 16381410 | 0.34 | CNV |
| chr3 | 17219048 | 17274062 | 0.68 | CNV |
| chr3 | 17274062 | 17329262 | 0.31 | CNV |
| chr3 | 17329262 | 17385233 | 1.15 | CNV |
| chr3 | 20865957 | 20921989 | 0.16 | CNV |
| chr3 | 21032952 | 21087966 | 0.17 | CNV |
| chr3 | 25557115 | 25612129 | 0.40 | CNV |
| chr3 | 33574614 | 33629703 | 0.28 | CNV |
| chr3 | 79791521 | 79847120 | 0.19 | CNV |
| chr3 | 83195779 | 83250793 | 0.09 | CNV |
| chr3 | 93801331 | 93856344 | 0.23 | CNV |
| chr3 | 95140058 | 95195071 | 0.57 | CNV |
| chr3 | 114198213 | 114253226 | 0.21 | CNV |
| chr3 | 118152026 | 118207219 | 0.11 | CNV |
| chr3 | 120506908 | 120561922 | 0.43 | CNV |
| chr3 | 126061157 | 126116748 | 0.58 | CNV |
| chr3 | 127943109 | 127998123 | 0.16 | CNV |
| chr3 | 132387621 | 132442634 | 0.32 | CNV |
| chr3 | 133853356 | 133908546 | 0.70 | CNV |
| chr3 | 134571663 | 134626677 | 0.22 | CNV |
| chr4 | 48460271 | 48515288 | 0.12 | CNV |
| chr5 | 74227459 | 74282476 | 0.32 | CNV |
| chr5 | 76085145 | 76140306 | 0.21 | CNV |
| chr5 | 88453742 | 88509913 | 0.39 | CNV |
| chr5 | 88620758 | 88675798 | 0.28 | CNV |
| chr5 | 89065777 | 89121410 | 0.66 | CNV |
| chr5 | 91416029 | 91471350 | 0.31 | CNV |
| chr5 | 100276864 | 100333562 | 0.28 | CNV |
| chr5 | 100846235 | 100902722 | 0.52 | CNV |
| chr5 | 119609521 | 119669349 | 0.30 | CNV |
| chr5 | 141027309 | 141082435 | 0.16 | CNV |
| chr5 | 159108604 | 159164770 | 0.29 | CNV |
| chr5 | 168785582 | 168840695 | 0.27 | CNV |
| chr6 | 30714865 | 30769981 | 0.26 | CNV |
| chr6 | 89726033 | 89781044 | 0.25 | CNV |
| chr6 | 113037143 | 113092154 | 0.21 | CNV |
| chr6 | 114051301 | 114106661 | 0.23 | CNV |
| chr7 | 33722510 | 33777524 | 0.23 | CNV |
| chr7 | 50495368 | 50550989 | 0.38 | CNV |
| chr7 | 78878213 | 78933227 | 0.07 | CNV |
| chr7 | 82762404 | 82817418 | 0.42 | CNV |
| chr7 | 90393418 | 90450825 | 0.26 | CNV |
| chr7 | 91974857 | 92030112 | 0.46 | CNV |
| chr7 | 92085127 | 92140244 | 0.17 | CNV |
| chr7 | 94038094 | 94093108 | 0.09 | CNV |
| chr7 | 156771135 | 156826267 | 0.26 | CNV |
| chr8 | 18046165 | 18102156 | 0.45 | CNV |
| chr8 | 18712898 | 18768441 | 0.38 | CNV |
| chr8 | 19043822 | 19099614 | 0.41 | CNV |
| chr8 | 19099614 | 19154637 | 0.68 | CNV |
| chr8 | 29862823 | 29917867 | 0.30 | CNV |
| chr9 | 759642 | 814678 | 0.24 | CNV |
| chr9 | 6053160 | 6109673 | 0.22 | CNV |
| chr9 | 7557960 | 7612969 | 0.12 | CNV |
| chr9 | 9445177 | 9500485 | 0.17 | CNV |
| chr9 | 11675419 | 11731402 | 1.24 | CNV |
| chr9 | 13848828 | 13903912 | 0.23 | CNV |
| chr9 | 17073502 | 17128511 | 0.33 | CNV |
| chr9 | 19153944 | 19209015 | 0.30 | CNV |
| chr9 | 19374362 | 19429757 | 0.26 | CNV |
| chr9 | 22179983 | 22236087 | 0.39 | CNV |
| chr9 | 22236087 | 22291096 | 0.15 | CNV |
| chr9 | 22517959 | 22574559 | 0.24 | CNV |
| chr9 | 79242352 | 79302027 | 0.15 | CNV |
| chr9 | 83445023 | 83500063 | 0.28 | CNV |
| chr9 | 83999459 | 84057600 | 0.12 | CNV |
| chr9 | 86565707 | 86620772 | 0.30 | CNV |
| chr9 | 100682639 | 100738188 | 0.38 | CNV |
| chr9 | 103520037 | 103578188 | 0.31 | CNV |
| chr9 | 111178070 | 111233825 | 0.30 | CNV |
| chr9 | 114690622 | 114745631 | 0.13 | CNV |
| chr9 | 131605064 | 131660148 | 0.13 | CNV |
| chr9 | 131990546 | 132045910 | 0.36 | CNV |
| chr9 | 132375985 | 132430994 | 0.37 | CNV |
| chr9 | 132486029 | 132541038 | 1.55 | CNV |
| chr9 | 132706065 | 132761103 | 0.27 | CNV |
| chr9 | 134236671 | 134291680 | 0.45 | CNV |
| chr9 | 137016185 | 137121821 | 0.18 | CNV |
| chr10 | 99124154 | 99179589 | 0.23 | CNV |
| chr10 | 104976790 | 105031807 | 0.94 | CNV |
| chr11 | 2417979 | 2473007 | 0.23 | CNV |
| chr11 | 3857332 | 3912361 | 0.27 | CNV |
| chr11 | 9120658 | 9175687 | 0.31 | CNV |
| chr11 | 9230715 | 9286071 | 0.76 | CNV |
| chr11 | 9341099 | 9396135 | 0.61 | CNV |
| chr11 | 10400422 | 10456702 | 0.12 | CNV |
| chr11 | 12667207 | 12722273 | 0.26 | CNV |
| chr11 | 13496640 | 13554507 | 0.27 | CNV |
| chr11 | 13613079 | 13669959 | 0.32 | CNV |
| chr11 | 18639832 | 18696667 | 1.68 | CNV |
| chr11 | 24117263 | 24172291 | 0.49 | CNV |
| chr11 | 29387297 | 29447009 | 0.43 | CNV |
| chr11 | 34405678 | 34460706 | 0.49 | CNV |
| chr11 | 36186788 | 36241985 | 0.20 | CNV |
| chr11 | 39367203 | 39423224 | 0.74 | CNV |
| chr11 | 47932469 | 47987497 | 0.43 | CNV |
| chr11 | 61783947 | 61838986 | 0.16 | CNV |
| chr14 | 48906309 | 48964351 | 0.45 | CNV |
| chr14 | 74248645 | 74303679 | 0.29 | CNV |
| chr14 | 75629599 | 75684915 | 0.37 | CNV |
| chr14 | 77397316 | 77452350 | 0.25 | CNV |
| chr15 | 41503949 | 41558961 | 0.28 | CNV |
| chr15 | 90673543 | 90728556 | 0.14 | CNV |
| chr16 | 3264192 | 3319220 | 0.22 | CNV |
| chr16 | 9118787 | 9173804 | 0.20 | CNV |
| chr17 | 1572640 | 1628296 | 0.33 | CNV |
| chr17 | 2460591 | 2515605 | 0.22 | CNV |
| chr17 | 2680657 | 2735671 | 0.31 | CNV |
| chr17 | 4298655 | 4353669 | 0.36 | CNV |
| chr17 | 6740035 | 6796661 | 0.33 | CNV |
| chr17 | 7460247 | 7516081 | 0.23 | CNV |
| chr17 | 8066899 | 8122046 | 0.52 | CNV |
| chr17 | 9891379 | 9948677 | 0.22 | CNV |
| chr17 | 10114028 | 10169050 | 0.35 | CNV |
| chr17 | 10279672 | 10334927 | 0.25 | CNV |
| chr17 | 14680777 | 14735935 | 0.71 | CNV |
| chr17 | 16249719 | 16305092 | 0.16 | CNV |
| chr17 | 70767592 | 70822606 | 0.33 | CNV |
| chr18 | 13215905 | 13270944 | 0.18 | CNV |
| chr18 | 55368140 | 55428127 | 0.30 | CNV |
| chr18 | 63218705 | 63274709 | 0.31 | CNV |
| chr19 | 10786103 | 10841103 | 0.21 | CNV |
| chr19 | 11391585 | 11447067 | 0.24 | CNV |
| chr19 | 13007338 | 13062338 | 0.14 | CNV |
| chr19 | 18434081 | 18489080 | 0.30 | CNV |
| chr19 | 32533120 | 32588119 | 0.22 | CNV |
| chr19 | 38835452 | 38890748 | 0.34 | CNV |
| chr19 | 58545142 | 58600142 | 0.15 | CNV |
| chr20 | 13365657 | 13421655 | 0.52 | CNV |
| chr20 | 20469497 | 20524543 | 0.21 | CNV |
| chr21 | 20631375 | 20686435 | 0.13 | CNV |
| chr22 | 36780005 | 36835591 | 0.28 | CNV |

TABLE 2
Urothelial Cancer-vs-Renal Cancer

| | Starting | Termination | | |
|---|---|---|---|---|
| Chromosome | Site | Site | Importance | Type |
| chr1 | 115212618 | 115212659 | 1.85 | MHB |
| chr11 | 14666887 | 14667109 | 0.52 | MHB |
| chr13 | 114518802 | 114518814 | 0.83 | MHB |
| chr13 | 73615586 | 73615695 | 0.85 | MHB |
| chr17 | 76886714 | 76886754 | 0.54 | MHB |
| chr4 | 161774249 | 161774454 | 0.39 | MHB |
| chr5 | 39188109 | 39188163 | 0.56 | MHB |
| chr6 | 26698208 | 26698231 | 0.55 | MHB |
| chr1 | 236129610 | 236129750 | 1.96 | MHB |
| chr10 | 23529521 | 23529557 | 1.00 | MHB |
| chr13 | 114475074 | 114475265 | 0.99 | MHB |
| chr15 | 48937065 | 48937117 | 0.72 | MHB |
| chr16 | 13184552 | 13184703 | 0.73 | MHB |
| chr16 | 85482572 | 85482600 | 0.74 | MHB |
| chr19 | 4089228 | 4089390 | 0.97 | MHB |
| chr2 | 188309968 | 188310077 | 1.10 | MHB |
| chr2 | 220417545 | 220417581 | 0.88 | MHB |
| chr2 | 241623230 | 241623242 | 0.94 | MHB |
| chr8 | 14336069 | 14336222 | 0.82 | MHB |
| chr8 | 144684401 | 144684454 | 1.29 | MHB |
| chr1 | 48844851 | 48902388 | 0.88 | CNV |
| chr1 | 174308449 | 174371408 | 0.86 | CNV |
| chr1 | 178685501 | 178740526 | 0.38 | CNV |
| chr2 | 234806969 | 234862038 | 0.63 | CNV |
| chr3 | 15771733 | 15827998 | 0.47 | CNV |
| chr3 | 16990918 | 17051090 | 1.12 | CNV |
| chr3 | 17607939 | 17662975 | 0.90 | CNV |
| chr3 | 23275728 | 23332367 | 0.64 | CNV |
| chr3 | 95195071 | 95250400 | 0.44 | CNV |
| chr3 | 111903356 | 111961403 | 0.48 | CNV |
| chr3 | 113475577 | 113531126 | 0.50 | CNV |
| chr3 | 121574590 | 121630757 | 0.59 | CNV |
| chr3 | 138183257 | 138238340 | 0.53 | CNV |
| chr3 | 139299812 | 139358167 | 0.64 | CNV |
| chr3 | 174301890 | 174359473 | 0.39 | CNV |
| chr5 | 62176803 | 62231839 | 0.92 | CNV |
| chr5 | 66487584 | 66544147 | 0.60 | CNV |
| chr5 | 121234184 | 121290948 | 0.45 | CNV |
| chr5 | 123864433 | 123919529 | 0.65 | CNV |
| chr5 | 147102018 | 147157035 | 0.49 | CNV |
| chr5 | 147157035 | 147212703 | 0.57 | CNV |
| chr5 | 152604120 | 152659617 | 1.01 | CNV |
| chr5 | 163462301 | 163517393 | 0.73 | CNV |
| chr5 | 163904265 | 163960432 | 0.72 | CNV |
| chr5 | 164570122 | 164625239 | 0.67 | CNV |
| chr5 | 165902828 | 165957845 | 0.70 | CNV |
| chr6 | 113037143 | 113092154 | 0.69 | CNV |
| chr7 | 87639055 | 87694465 | 0.94 | CNV |
| chr8 | 24357563 | 24412586 | 0.53 | CNV |
| chr8 | 24470110 | 24525132 | 0.66 | CNV |
| chr8 | 26083221 | 26138274 | 1.48 | CNV |
| chr8 | 29807800 | 29862823 | 0.70 | CNV |
| chr8 | 74566649 | 74622318 | 0.85 | CNV |
| chr8 | 84671826 | 84726867 | 0.68 | CNV |
| chr9 | 7281976 | 7336999 | 0.41 | CNV |
| chr9 | 21396337 | 21451882 | 0.48 | CNV |
| chr9 | 83556168 | 83611242 | 0.62 | CNV |
| chr10 | 109201863 | 109260402 | 0.59 | CNV |
| chr10 | 115516012 | 115571210 | 0.58 | CNV |
| chr11 | 24117263 | 24172291 | 0.57 | CNV |
| chr11 | 29107719 | 29162747 | 0.34 | CNV |
| chr11 | 105083339 | 105138374 | 0.86 | CNV |
| chr11 | 122263376 | 122318578 | 0.44 | CNV |
| chr14 | 71290234 | 71345702 | 0.37 | CNV |
| chr17 | 10224263 | 10279672 | 0.50 | CNV |
| chr17 | 10446415 | 10501971 | 0.93 | CNV |
| chr17 | 77891317 | 77946332 | 0.60 | CNV |
| chr3 | 17441590 | 17496795 | 0.67 | CNV |
| chr3 | 17718745 | 17777075 | 1.28 | CNV |
| chr3 | 107302517 | 107357531 | 1.31 | CNV |
| chr3 | 113641548 | 113696867 | 0.66 | CNV |
| chr3 | 130811969 | 130868365 | 1.17 | CNV |
| chr3 | 133853356 | 133908546 | 1.15 | CNV |
| chr4 | 167158821 | 167216216 | 0.49 | CNV |
| chr5 | 89121410 | 89176427 | 1.07 | CNV |
| chr5 | 122753969 | 122810170 | 0.76 | CNV |
| chr5 | 162069225 | 162125520 | 1.28 | CNV |
| chr6 | 153978920 | 154034743 | 0.76 | CNV |
| chr8 | 15322023 | 15377045 | 0.64 | CNV |
| chr8 | 18102156 | 18157179 | 1.00 | CNV |
| chr8 | 19043822 | 19099614 | 0.88 | CNV |
| chr8 | 24076615 | 24134608 | 0.55 | CNV |
| chr8 | 26028199 | 26083221 | 1.18 | CNV |
| chr8 | 93887300 | 93942322 | 1.17 | CNV |
| chr9 | 76347301 | 76402310 | 1.17 | CNV |
| chr9 | 100682639 | 100738188 | 0.58 | CNV |
| chr9 | 117452632 | 117507877 | 0.97 | CNV |
| chr10 | 86724476 | 86780320 | 0.71 | CNV |
| chr10 | 95612934 | 95667951 | 0.85 | CNV |
| chr10 | 101767751 | 101822768 | 1.00 | CNV |
| chr10 | 110379163 | 110434302 | 0.89 | CNV |
| chr11 | 40319502 | 40374531 | 0.87 | CNV |
| chr11 | 40931292 | 40989227 | 1.54 | CNV |
| chr11 | 114212102 | 114267174 | 0.52 | CNV |
| chr17 | 15288166 | 15343262 | 0.61 | CNV |
| chr17 | 61092762 | 61147777 | 0.70 | CNV |
| chr19 | 35079100 | 35136146 | 0.85 | CNV |
| chr19 | 35136146 | 35191864 | 2.12 | CNV |

TABLE 3
Urothelial Cancer-vs-Prostate Cancer

| | Starting | Termination | | |
|---|---|---|---|---|
| Chromosome | Site | Site | Importance | Type |
| chr1 | 12203871 | 12203905 | 0.573298 | MHB |
| chr1 | 15743670 | 15743692 | 1.542805 | MHB |
| chr1 | 219634296 | 219634397 | 0.934587 | MHB |
| chr1 | 31230080 | 31230098 | 0.878825 | MHB |
| chr1 | 67195043 | 67195190 | 0.977256 | MHB |
| chr10 | 11183275 | 11183349 | 0.484171 | MHB |
| chr10 | 121030613 | 121030662 | 0.782292 | MHB |
| chr10 | 121441698 | 121441880 | 1.168434 | MHB |
| chr10 | 12490843 | 12490884 | 2.08052 | MHB |
| chr10 | 135088522 | 135088585 | 0.366013 | MHB |
| chr11 | 129150328 | 129150359 | 1.003039 | MHB |
| chr11 | 16023703 | 16023848 | 0.349497 | MHB |
| chr11 | 47236650 | 47236864 | 0.41618 | MHB |
| chr13 | 27565252 | 27565508 | 0.822781 | MHB |
| chr14 | 100535084 | 100535221 | 1.076337 | MHB |
| chr14 | 22896829 | 22896869 | 0.659218 | MHB |
| chr14 | 79502927 | 79503069 | 0.568811 | MHB |
| chr15 | 38422144 | 38422197 | 0.754938 | MHB |
| chr16 | 80840916 | 80840984 | 0.758611 | MHB |
| chr17 | 38703765 | 38703933 | 0.904598 | MHB |
| chr17 | 38738716 | 38738723 | 1.465557 | MHB |
| chr17 | 73840350 | 73840387 | 0.43623 | MHB |
| chr17 | 7482474 | 7482694 | 0.248248 | MHB |
| chr19 | 19083036 | 19083146 | 0.847764 | MHB |
| chr19 | 42703701 | 42703778 | 0.762135 | MHB |
| chr2 | 102187418 | 102187570 | 1.208225 | MHB |
| chr2 | 103353211 | 103353278 | 0.428877 | MHB |
| chr2 | 109952264 | 109952432 | 0.891393 | MHB |
| chr2 | 120934486 | 120934649 | 1.248863 | MHB |
| chr2 | 196401030 | 196401147 | 0.478669 | MHB |
| chr2 | 20624586 | 20624757 | 1.269155 | MHB |
| chr2 | 219866511 | 219866527 | 0.496729 | MHB |
| chr2 | 227001592 | 227001693 | 0.658201 | MHB |
| chr2 | 236299222 | 236299346 | 0.685026 | MHB |
| chr2 | 238582223 | 238582238 | 0.40122 | MHB |
| chr2 | 65593907 | 65593933 | 0.501373 | MHB |
| chr2 | 80221460 | 80221514 | 0.432 | MHB |
| chr20 | 46115992 | 46116225 | 1.152386 | MHB |
| chr20 | 50618683 | 50618811 | 2.227494 | MHB |
| chr21 | 39850738 | 39850916 | 1.258264 | MHB |
| chr21 | 40386819 | 40386913 | 0.905596 | MHB |
| chr22 | 29810912 | 29811014 | 0.644997 | MHB |
| chr3 | 176919546 | 176919570 | 1.093437 | MHB |
| chr3 | 37500143 | 37500244 | 0.431962 | MHB |
| chr3 | 38468403 | 38468436 | 1.27045 | MHB |
| chr3 | 59413091 | 59413193 | 0.898936 | MHB |
| chr3 | 71493368 | 71493587 | 0.760574 | MHB |
| chr4 | 186818095 | 186818294 | 1.203058 | MHB |
| chr4 | 66764752 | 66764870 | 0.779961 | MHB |
| chr4 | 78508318 | 78508537 | 1.596627 | MHB |
| chr5 | 32774736 | 32774858 | 0.505749 | MHB |
| chr5 | 43039406 | 43039412 | 0.764542 | MHB |
| chr5 | 81653162 | 81653356 | 1.914996 | MHB |
| chr6 | 146679333 | 146679448 | 0.766335 | MHB |
| chr7 | 145452125 | 145452184 | 1.430398 | MHB |
| chr7 | 17274287 | 17274420 | 0.812012 | MHB |
| chr7 | 5437106 | 5437149 | 0.604728 | MHB |
| chr8 | 116457980 | 116458111 | 0.278563 | MHB |
| chr8 | 37595362 | 37595410 | 0.29335 | MHB |
| chr8 | 40625223 | 40625323 | 0.206973 | MHB |
| chr8 | 87520493 | 87520578 | 0.538615 | MHB |
| chr8 | 99478792 | 99478938 | 1.007536 | MHB |
| chr9 | 129748188 | 129748241 | 1.242409 | MHB |
| chr2 | 10548492 | 10548671 | 0.890197 | MHB |
| chr1 | 159961820 | 160016845 | 0.377615 | CNV |
| chr1 | 161743453 | 161798982 | 0.42517 | CNV |
| chr1 | 162076340 | 162131365 | 0.416175 | CNV |
| chr1 | 162521424 | 162576449 | 0.446689 | CNV |
| chr1 | 162686499 | 162744694 | 0.422095 | CNV |
| chr2 | 209033619 | 209089253 | 0.212744 | CNV |
| chr2 | 232667479 | 232738358 | 0.267648 | CNV |
| chr2 | 233919413 | 233974434 | 0.29052 | CNV |
| chr5 | 56853464 | 56908701 | 0.329268 | CNV |
| chr5 | 57753633 | 57808650 | 0.189869 | CNV |
| chr5 | 74227459 | 74282476 | 0.393206 | CNV |
| chr5 | 81174138 | 81229564 | 0.288613 | CNV |
| chr5 | 88453742 | 88509913 | 0.349771 | CNV |
| chr5 | 88620758 | 88675798 | 0.256986 | CNV |
| chr5 | 89121410 | 89176427 | 0.274956 | CNV |
| chr5 | 89629928 | 89684945 | 0.441073 | CNV |
| chr5 | 130289101 | 130344747 | 0.463226 | CNV |
| chr5 | 133359302 | 133414319 | 0.332077 | CNV |
| chr5 | 141912654 | 141967709 | 0.373973 | CNV |
| chr5 | 151909863 | 151965045 | 0.276912 | CNV |
| chr5 | 160448299 | 160506802 | 0.273978 | CNV |
| chr5 | 164735273 | 164790598 | 0.690886 | CNV |
| chr5 | 164902179 | 164957196 | 0.546989 | CNV |
| chr5 | 165902828 | 165957845 | 0.497043 | CNV |
| chr5 | 166068795 | 166123812 | 0.678864 | CNV |
| chr5 | 166234782 | 166289800 | 1.549945 | CNV |
| chr5 | 174061726 | 174116744 | 0.464477 | CNV |
| chr5 | 174116744 | 174171761 | 0.127589 | CNV |
| chr5 | 175006048 | 175061065 | 0.196762 | CNV |
| chr6 | 20586708 | 20643594 | 0.344617 | CNV |
| chr6 | 21030108 | 21085119 | 1.485941 | CNV |
| chr7 | 93197341 | 93256442 | 0.335725 | CNV |
| chr7 | 94589338 | 94644853 | 0.330557 | CNV |
| chr9 | 32172294 | 32233261 | 0.296578 | CNV |
| chr9 | 131990546 | 132045910 | 0.362283 | CNV |
| chr10 | 94894732 | 94949750 | 0.48165 | CNV |
| chr10 | 110324146 | 110379163 | 0.282774 | CNV |
| chr10 | 120953429 | 121008446 | 0.309671 | CNV |
| chr11 | 10677931 | 10733225 | 0.231444 | CNV |
| chr11 | 10733225 | 10788253 | 0.311036 | CNV |
| chr11 | 22880479 | 22937529 | 0.391737 | CNV |
| chr11 | 27761188 | 27816888 | 0.38865 | CNV |
| chr11 | 39423224 | 39478253 | 0.356691 | CNV |
| chr11 | 113825661 | 113880690 | 0.411814 | CNV |
| chr11 | 115437817 | 115493057 | 0.41799 | CNV |
| chr11 | 118049482 | 118104510 | 0.485633 | CNV |
| chr17 | 7956805 | 8011885 | 0.344418 | CNV |

TABLE 4
Renal Cancer-vs-Healthy

| | Starting | Termination | | |
|---|---|---|---|---|
| Chromosome | Site | Site | Importance | Type |
| chr10 | 102242528 | 102242543 | 1.80 | MHB |
| chr10 | 21814384 | 21814394 | 1.47 | MHB |
| chr11 | 10829574 | 10829619 | 2.42 | MHB |
| chr17 | 7382578 | 7382823 | 1.61 | MHB |
| chr19 | 13617083 | 13617103 | 1.38 | MHB |
| chr19 | 36347379 | 36347453 | 1.68 | MHB |
| chr2 | 169746957 | 169746975 | 1.56 | MHB |
| chr5 | 174151629 | 174151637 | 1.10 | MHB |
| chr6 | 97345724 | 97345780 | 3.47 | MHB |
| chr7 | 122526931 | 122526958 | 1.96 | MHB |
| chr7 | 130791008 | 130791082 | 1.84 | MHB |
| chr1 | 33646761 | 33646778 | 1.18 | MHB |
| chr14 | 24610178 | 24610249 | 1.36 | MHB |
| chr19 | 54982794 | 54982803 | 1.59 | MHB |
| chr5 | 94956094 | 94956112 | 3.20 | MHB |
| chr6 | 17102376 | 17102462 | 2.39 | MHB |
| chr8 | 637408 | 637421 | 2.62 | MHB |
| chr3 | 116003 | 171017 | 2.42 | CNV |
| chr3 | 25557115 | 25612129 | 1.25 | CNV |
| chr5 | 16085363 | 16140380 | 2.43 | CNV |
| chr5 | 74506474 | 74562673 | 2.07 | CNV |
| chr5 | 152889285 | 152944303 | 3.67 | CNV |
| chr5 | 159937141 | 159992174 | 2.28 | CNV |
| chr6 | 99690430 | 99745774 | 3.02 | CNV |
| chr7 | 8513355 | 8568522 | 2.70 | CNV |
| chr7 | 11247739 | 11302753 | 2.73 | CNV |
| chr7 | 132285752 | 132340767 | 2.10 | CNV |
| chr9 | 33813192 | 33868200 | 3.18 | CNV |
| chr9 | 108447776 | 108503338 | 2.72 | CNV |
| chr9 | 110735342 | 110791265 | 2.70 | CNV |
| chr14 | 53355525 | 53410559 | 2.18 | CNV |
| chr14 | 64126542 | 64181576 | 2.50 | CNV |
| chr14 | 103847457 | 103902492 | 3.69 | CNV |

TABLE 5
Renal Cancer-vs-Prostate Cancer

| | Starting | Termination | | |
|---|---|---|---|---|
| Chromosome | Site | Site | Importance | Type |
| chr1 | 22109859 | 22109916 | 0.87 | MHB |
| chr10 | 49497933 | 49498073 | 1.14 | MHB |
| chr12 | 77719371 | 77719416 | 0.95 | MHB |
| chr15 | 86186010 | 86186094 | 0.78 | MHB |
| chr16 | 1993426 | 1993506 | 0.68 | MHB |
| chr17 | 40718872 | 40719166 | 1.11 | MHB |
| chr19 | 35451370 | 35451530 | 0.77 | MHB |
| chr19 | 49652993 | 49653046 | 1.22 | MHB |
| chr2 | 186289811 | 186289826 | 0.92 | MHB |
| chr3 | 190580534 | 190580736 | 0.60 | MHB |
| chr5 | 58335019 | 58335266 | 1.07 | MHB |
| chr5 | 74616662 | 74616884 | 0.62 | MHB |
| chr6 | 136571049 | 136571096 | 1.15 | MHB |
| chr6 | 34111804 | 34112020 | 1.38 | MHB |
| chr6 | 44225065 | 44225303 | 1.22 | MHB |
| chr1 | 152627727 | 152627921 | 1.34 | MHB |
| chr1 | 180198441 | 180198461 | 0.88 | MHB |
| chr11 | 62691233 | 62691294 | 0.95 | MHB |
| chr12 | 120988038 | 120988152 | 0.63 | MHB |
| chr13 | 53024417 | 53024656 | 1.15 | MHB |
| chr14 | 102247976 | 102248130 | 1.10 | MHB |
| chr15 | 55560030 | 55560060 | 1.84 | MHB |
| chr16 | 3097024 | 3097094 | 1.02 | MHB |
| chr16 | 745584 | 745614 | 0.77 | MHB |
| chr18 | 13218404 | 13218646 | 1.35 | MHB |
| chr19 | 1546205 | 1546320 | 0.91 | MHB |
| chr2 | 10548492 | 10548671 | 1.02 | MHB |
| chr2 | 120027340 | 120027429 | 1.10 | MHB |
| chr20 | 47426191 | 47426375 | 1.21 | MHB |
| chr20 | 52566006 | 52566098 | 1.06 | MHB |
| chr22 | 22337255 | 22337322 | 0.89 | MHB |
| chr3 | 38480046 | 38480221 | 0.72 | MHB |
| chr5 | 176882950 | 176883082 | 1.07 | MHB |
| chr7 | 105447174 | 105447254 | 1.20 | MHB |
| chr9 | 109722717 | 109722878 | 1.16 | MHB |
| chr4 | 66201793 | 66257548 | 0.69 | CNV |
| chr4 | 94301267 | 94356284 | 0.95 | CNV |
| chr4 | 150299188 | 150354458 | 0.81 | CNV |
| chr4 | 167158821 | 167216216 | 1.29 | CNV |
| chr4 | 167902207 | 167957223 | 0.86 | CNV |
| chr5 | 146433589 | 146488606 | 0.83 | CNV |
| chr6 | 113037143 | 113092154 | 1.05 | CNV |
| chr6 | 153978920 | 154034743 | 0.84 | CNV |
| chr7 | 111600515 | 111661508 | 1.02 | CNV |
| chr9 | 28875243 | 28931385 | 0.79 | CNV |
| chr9 | 81468449 | 81526520 | 0.97 | CNV |
| chr9 | 117618821 | 117673887 | 0.90 | CNV |
| chr9 | 121338520 | 121393528 | 1.45 | CNV |
| chr11 | 80465398 | 80521201 | 0.77 | CNV |
| chr11 | 80576229 | 80631258 | 0.88 | CNV |
| chr11 | 105083339 | 105138374 | 0.81 | CNV |
| chr11 | 121876562 | 121932445 | 0.98 | CNV |
| chr12 | 29623425 | 29678433 | 1.14 | CNV |
| chr13 | 37757179 | 37812571 | 1.20 | CNV |
| chr13 | 50446332 | 50501664 | 1.19 | CNV |
| chr13 | 50501664 | 50556692 | 0.78 | CNV |
| chr14 | 41642756 | 41703315 | 0.66 | CNV |
| chr15 | 40785196 | 40840208 | 0.79 | CNV |
| chr15 | 50635023 | 50690035 | 0.50 | CNV |
| chr15 | 50965628 | 51020959 | 1.04 | CNV |
| chr21 | 24781347 | 24836631 | 1.05 | CNV |
| chr21 | 37747140 | 37802429 | 0.88 | CNV |
| chr21 | 47716829 | 47772101 | 0.96 | CNV |

TABLE 6
Prostate Cancer-vs-Healthy

| | Starting | Termination | | |
|---|---|---|---|---|
| Chromosome | Site | Site | Importance | Type |
| chr17 | 27347046 | 27347060 | 2.96 | MHB |
| chr19 | 37861958 | 37862007 | 3.70 | MHB |
| chr2 | 44973114 | 44973313 | 3.68 | MHB |
| chr3 | 111698032 | 111698142 | 2.79 | MHB |
| chr3 | 171527304 | 171527450 | 1.17 | MHB |
| chr7 | 155598358 | 155598674 | 1.96 | MHB |
| chr7 | 2281362 | 2281400 | 2.63 | MHB |
| chr8 | 146228339 | 146228379 | 2.99 | MHB |
| chr1 | 32827699 | 32827730 | 2.68 | MHB |
| chr10 | 53248433 | 53248618 | 1.60 | MHB |
| chr15 | 32639333 | 32639373 | 3.44 | MHB |
| chr18 | 55108538 | 55108557 | 2.37 | MHB |
| chr19 | 41857573 | 41857626 | 3.62 | MHB |
| chr2 | 197962551 | 197962721 | 2.65 | MHB |
| chr3 | 71493368 | 71493587 | 1.73 | MHB |
| chr7 | 27202221 | 27202344 | 2.40 | MHB |
| chr9 | 32573142 | 32573226 | 2.81 | MHB |
| chr5 | 77254908 | 77309925 | 0.90 | CNV |
| chr6 | 72575407 | 72630418 | 1.34 | CNV |
| chr6 | 84711070 | 84766081 | 0.89 | CNV |
| chr6 | 108913361 | 108968372 | 1.15 | CNV |
| chr8 | 18433893 | 18490889 | 1.93 | CNV |
| chr8 | 70880037 | 70935097 | 1.15 | CNV |
| chr8 | 70935097 | 70990165 | 1.02 | CNV |
| chr8 | 90752002 | 90807056 | 0.96 | CNV |
| chr8 | 102606158 | 102661373 | 1.35 | CNV |
| chr8 | 139706847 | 139762642 | 0.74 | CNV |
| chr12 | 14517394 | 14572402 | 0.84 | CNV |
| chr13 | 35476446 | 35531620 | 0.86 | CNV |
| chr13 | 53392087 | 53447404 | 1.27 | CNV |
| chr13 | 61442988 | 61498016 | 0.94 | CNV |
| chr16 | 63917832 | 63972849 | 0.73 | CNV |
| chr18 | 26763015 | 26818814 | 0.92 | CNV |
| chr18 | 30035947 | 30090986 | 0.84 | CNV |
| chr18 | 31704358 | 31761546 | 0.67 | CNV |
| chr18 | 45420426 | 45475465 | 0.88 | CNV |
| chr18 | 46415737 | 46470811 | 1.06 | CNV |
| chr18 | 46919903 | 46976529 | 1.36 | CNV |
| chr18 | 60879561 | 60934690 | 0.62 | CNV |
| chr18 | 63163316 | 63218705 | 0.80 | CNV |
| chr18 | 68952678 | 69008529 | 0.77 | CNV |
| chr18 | 69342463 | 69397502 | 1.04 | CNV |
| chr18 | 69898028 | 69953299 | 0.78 | CNV |

F5 represented the features required for a hybrid model for integrating DNA methylation and copy number information, and the classification model constructed with F5 performs the best. In this way, the binary classification model was established.

This model can be used to distinguish tumor patients from healthy people.

As previously described, the inventors collected 100 samples of urothelial cancer (UC) (including bladder cancer and upper tract urothelial cancer), 65 samples of kidney renal clear cell carcinoma (KIRC) and 60 samples of prostate cancer (PRAD), and 88 samples of healthy people. Each sample included the feature information of F1 to F5. Taking the UC-vs-Healthy binary classifier as an example, the samples were first randomly rearranged so that the composite matrix of the samples had no preference, and then was split into a training set and a test set according to a ration of 5:1. Next, modeling was performed using the above-screened features (e.g., F5) combined with a support vector machine algorithm. Then, the test set was used to test the model performance, including accuracy, sensitivity, specificity, AUC and Kappa value. The above process was repeated 10 times, and the average accuracy, sensitivity, specificity, area under the curve (AUC) and Kappa coefficient of the ten results represented the stable classification performance of a binary classifier of urothelial cancer-vs-healthy. Other binary classifiers (Renal Cancer-vs-Healthy, Prostate Cancer-vs-Healthy) were constructed in a similar way.

The results were shown in Table 7 below.

TABLE 7

| | | Area | | | | |
|---|---|---|---|---|---|---|
| Feature | | Under | Kappa | | | |
| Type | Accuracy | Curve | Value | Sensitivity | Specificity | Binary Classifier Type |
| f1 | 0.900 | 0.952 | 0.798 | 0.929 | 0.867 | urothelial cancer-vs-healthy |
| f2 | 0.950 | 0.992 | 0.899 | 0.982 | 0.913 | urothelial cancer-vs-healthy |
| f3 | 0.944 | 0.987 | 0.887 | 0.971 | 0.913 | urothelial cancer-vs-healthy |
| f4 | 0.931 | 0.984 | 0.863 | 0.918 | 0.947 | urothelial cancer-vs-healthy |
| f5 | 0.978 | 0.996 | 0.956 | 0.976 | 0.980 | urothelial cancer-vs-healthy |
| f1 | 0.823 | 0.907 | 0.641 | 0.827 | 0.820 | renal cancer-vs-healthy |
| f2 | 0.881 | 0.963 | 0.758 | 0.891 | 0.873 | renal cancer-vs-healthy |
| f3 | 0.919 | 0.958 | 0.833 | 0.882 | 0.947 | renal cancer-vs-healthy |
| f4 | 0.885 | 0.913 | 0.758 | 0.782 | 0.960 | renal cancer-vs-healthy |
| f5 | 0.938 | 0.967 | 0.874 | 0.918 | 0.953 | renal cancer-vs-healthy |
| f1 | 0.896 | 0.972 | 0.776 | 0.800 | 0.960 | prostate cancer -vs-healthy |
| f2 | 0.900 | 0.981 | 0.788 | 0.840 | 0.940 | prostate cancer-vs-healthy |
| f3 | 0.948 | 0.995 | 0.891 | 0.930 | 0.960 | prostate cancer-vs-healthy |
| f4 | 0.916 | 0.940 | 0.820 | 0.830 | 0.973 | prostate cancer-vs-healthy |
| f5 | 0.952 | 0.991 | 0.898 | 0.900 | 0.987 | prostate cancer-vs-healthy |
| f1 | 0.893 | 0.954 | 0.769 | 0.924 | 0.840 | urothelial cancer-vs-prostate |
| | | | | | | cancer |
| f2 | 0.930 | 0.978 | 0.847 | 0.953 | 0.890 | urothelial cancer-vs-prostate |
| | | | | | | cancer |
| f3 | 0.933 | 0.974 | 0.855 | 0.953 | 0.900 | urothelial cancer-vs-prostate |
| | | | | | | cancer |
| f4 | 0.915 | 0.982 | 0.819 | 0.924 | 0.900 | urothelial cancer-vs-prostate |
| | | | | | | cancer |
| f5 | 0.941 | 0.990 | 0.872 | 0.959 | 0.910 | urothelial cancer-vs-prostate |
| | | | | | | cancer |
| f1 | 0.786 | 0.810 | 0.526 | 0.888 | 0.627 | urothelial cancer-vs-renal |
| | | | | | | cancer |
| f2 | 0.864 | 0.931 | 0.695 | 0.941 | 0.745 | urothelial cancer-vs-renal |
| | | | | | | cancer |
| f3 | 0.896 | 0.920 | 0.764 | 0.965 | 0.791 | urothelial cancer-vs-renal |
| | | | | | | cancer |
| f4 | 0.850 | 0.909 | 0.666 | 0.924 | 0.736 | urothelial cancer-vs-renal |
| | | | | | | cancer |
| f5 | 0.879 | 0.922 | 0.725 | 0.953 | 0.764 | urothelial cancer-vs-renal |
| | | | | | | cancer |
| f1 | 0.943 | 0.983 | 0.885 | 0.955 | 0.930 | renal cancer-vs-prostate cancer |
| f2 | 0.971 | 0.994 | 0.943 | 0.973 | 0.970 | renal cancer-vs-prostate cancer |
| f3 | 0.948 | 0.996 | 0.895 | 0.964 | 0.930 | renal cancer-vs-prostate cancer |
| f4 | 0.762 | 0.902 | 0.521 | 0.800 | 0.720 | renal cancer-vs-prostate cancer |
| f5 | 0.938 | 0.977 | 0.877 | 0.909 | 0.970 | renal cancer-vs-prostate cancer |

The results showed that the accuracy of the 10-time repeated modeling and prediction of the corresponding classifier model was more than 90%. By feature selection and construction of the corresponding binary classifiers, the classifier model constructed by the inventors using the F5 features had the best performance, not only higher than the performance of the classifiers constructed only with DNA methylation information (F1, F2 and F3), but also higher than the performance of the classifier constructed with only DNA copy number information (F4).

### Example 8: Establishment and Validation of Tumor Tissue Typing Model (Multi-Stage Classifiers)

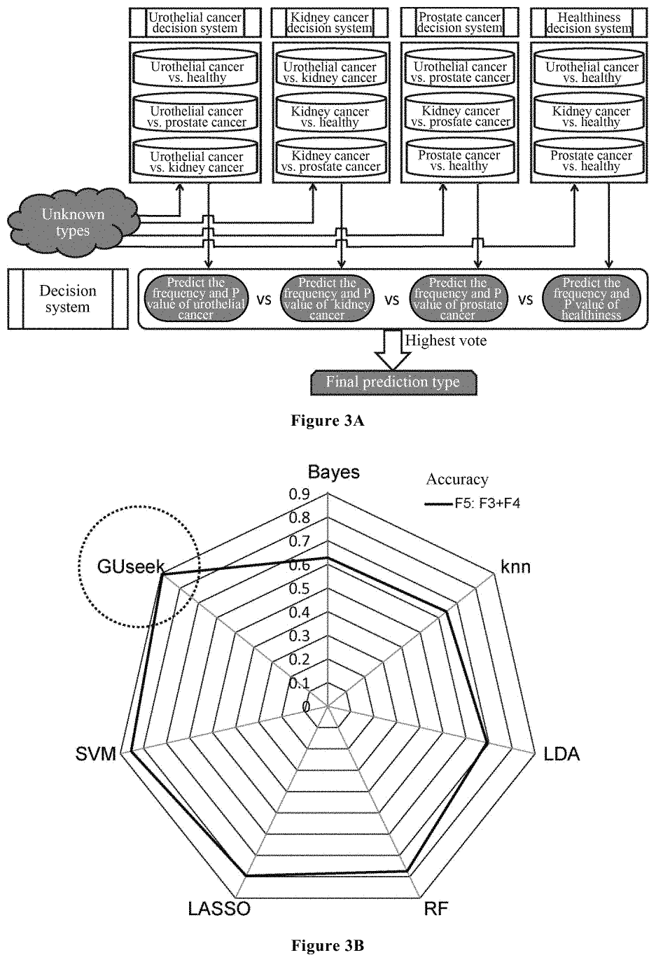

For the tumor tissue typing model, the inventors constructed a multi-stage classification model (named as genitourinary cancers seek, abbreviated as GUseek) based on binary classifier models (shown in FIG. 3A).

The main aim of GUseek was to differentiate urothelial cancer (UC) (including bladder cancer and upper tract urothelial cancer), kidney renal clear cell carcinoma (KIRC), and prostate cancer (PRAD).

Based on the binary classification concept, there were six sets of binary classifiers, i.e., urothelial cancer-vs-healthy, urothelial cancer-vs-renal cancer, urothelial cancer-vs-prostate cancer, renal cancer-vs-healthy, renal cancer-vs-prostate cancer, and prostate cancer-vs-healthy, which can be combined into four sets of classification decision systems, i.e.:

a urothelial cancer decision system (including urothelial cancer-vs-healthy, urothelial cancer-vs-renal cancer and urothelial cancer-vs-prostate cancer),

a renal cancer decision system (including urothelial cancer-vs-renal cancer, renal cancer-vs-healthy and renal cancer-vs-prostate cancer),

a prostate cancer decision system (including urothelial cancer-vs-prostate cancer, renal cancer-vs-prostate cancer and prostate cancer-vs-healthy), and

a healthiness decision system (including urothelial cancer-vs-healthy, renal cancer-vs-healthy and prostate cancer-vs-healthy).

An unknown sample was first mapped to each decision system for predictive analysis, and the proportion of the prediction category of each decision system was provided accordingly. By integrating the scores of various types in the four decision systems, the category with the highest score was defined as the prediction category of the unknown sample. If there was more than one category with the highest score, the category with the highest score probability was selected as the final prediction category for the unknown sample. Considering that it was theoretically impossible for a female to be predicted to have prostate cancer, if a female sample was predicted to be prostate cancer, a sub-optimal prediction result was taken. For example, if the vote predicted to be renal cancer was second only to prostate cancer, the predictive label of the female sample was defined as renal cancer. If the numbers of votes were the same, then the probabilities were compared. The category with higher probability was taken as the final prediction result of the female sample.

The GUseek model can use the advantages of binary classification to the maximum, while a more powerful multi-stage classifier can be constructed by integrating multiple machine learning algorithms. By integrating the SVM algorithm, the GUseek constructed by the inventors can achieve 10-time repeated modeling and prediction accuracy up to nearly 90% (89.43%). The specific method was as follows.

The present inventors first randomly rearranged the collected 100 samples of urothelial cancer (UC) (including bladder cancer and upper tract urothelial cancer), 65 samples of kidney renal clear cell carcinoma (KIRC) and 60 samples of prostate cancer (PRAD), and 88 samples of healthy people and split the samples into a training set and a test set according to a ratio of 5:1 (see Table 8).

TABLE 8

| | | Number of | Number of |
|---|---|---|---|
| | Number per | Subjects in | Subjects in |
| Subject Grouping | Group | Training Sets | Test Sets |
| Samples from healthy human | 88 | 73 | 15 |
| Samples from kidney renal | 65 | 54 | 11 |
| clear cell carcinoma patients | | | |
| Samples from urothelial | 100 | 83 | 17 |
| cancer patients | | | |
| Samples from prostate | 60 | 50 | 10 |
| cancer patients | | | |

Six sets of binary classifiers were then constructed according to the above method of constructing binary classifiers, and were further combined to form four decision systems. For each sample in the test set, prediction was first performed in the binary classifiers and corresponding prediction categories and probabilities were obtained according to the input requirements of the binary classifiers of individual decision systems. The category of the predicted sample was determined by comparing the predicted times (the number of votes) of the sample by individual decision systems. If the numbers of votes for determining the decision category were comparable, the corresponding probabilities were further compared, and the category with the highest probability was taken as the final prediction category of the sample. In this way, the inventors can finally obtain the prediction classification of each test set sample, and can further obtain the prediction overall accuracy and Kappa coefficient of the GUseek model by constructing a hybrid matrix. The above process was repeated 10 times, and the obtained average accuracy was the stability performance of the GUseek. See FIG. 3B.

Using the integration algorithm GUseek proposed by the inventors, GUseek showed very high accuracies in 10-time remodeling and predictions (10-time average reached 89.43%, see FIG. 3B). The integration algorithm GUseek was superior to conventional multi-stage classification algorithms, including support vector machines (SVM), randomForest (RF), Bayes, LASSO, linear discriminant dimension reduction algorithm (LDA), and K-nearest neighbor algorithm (knn).

First, the training set that had been split according to a ratio of 5:1 by the GUseek analysis process was modeled according to the above algorithm in sequence, and then model evaluation was performed by using the test set. The assessment result was demonstrated by a hybrid matrix. The comparison results of one random time were shown in Tables 9-10, and the ten-time average accuracy was shown in FIG. 3B.

TABLE 9

| | Actual types of samples | | | |
|---|---|---|---|---|
| GUseek (F5) | urothelial | | Prostate | Renal |
| Test data set | cancer | Healthy | cancer | cancer |
| Urothelial cancer | 16 | 0 | 1 | 3 |
| Healthy | 0 | 15 | 0 | 0 |
| Prostate cancer | 1 | 0 | 9 | 0 |
| Renal cancer | 0 | 0 | 0 | 8 |
| Sensitivity | 94.12% | 100.0% | 90.00% | 72.73% |
| Specificity | 88.89% | 100.0% | 97.67% | 100.0% |
| Post-equilibrium | 91.50% | 100.0% | 93.84% | 86.36% |
| accuracy | | | | |

| | |
|---|---|
| Kappa value | 87.11% |
| Overall accuracy | 90.57% |

TABLE 10

| | Actual types of samples | | | |
|---|---|---|---|---|
| SVM (F5) | urothelial | | Prostate | Renal |
| Test data set | cancer | Healthy | cancer | cancer |
| Urothelial cancer | 15 | 1 | 1 | 3 |
| Health | 0 | 14 | 1 | 1 |
| Prostate cancer | 0 | 0 | 8 | 0 |
| Renal cancer | 2 | 0 | 0 | 7 |
| Sensitivity | 88.24% | 93.33% | 80.00% | 63.64% |
| Specificity | 86.11% | 94.74% | 100.00% | 95.24% |
| Post-equilibrium | 87.17% | 94.04% | 90.00% | 79.44% |
| accuracy | | | | |

| | |
|---|---|
| Kappa value | 76.73% |
| Overall accuracy | 83.02% |

The algorithm developed by the present inventors can integrate the optimal conventional algorithm to achieve the optimal combination, i.e., each decision classification system, and can be constructed by selecting an algorithm with the best classification effect, which then can be combined into an overall optimal classification system.

### Example 9: Establishment and Validation of Prognostic Risk Model

Prognostic markers of bladder cancer and renal cancer were screened respectively by using available clinical data of TCGA. The specific steps were as follows.

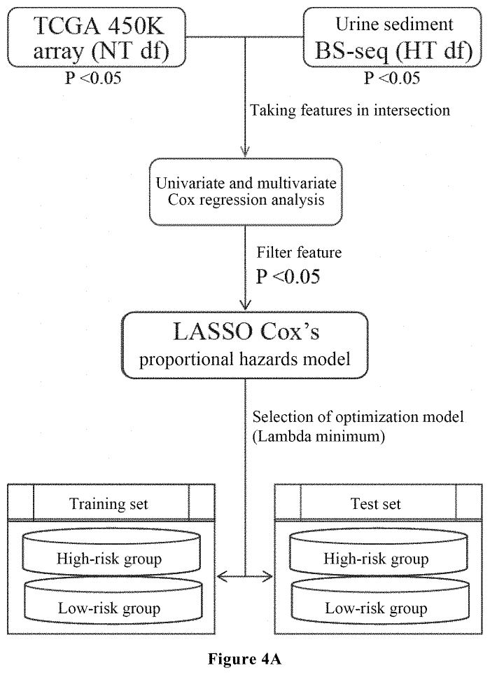

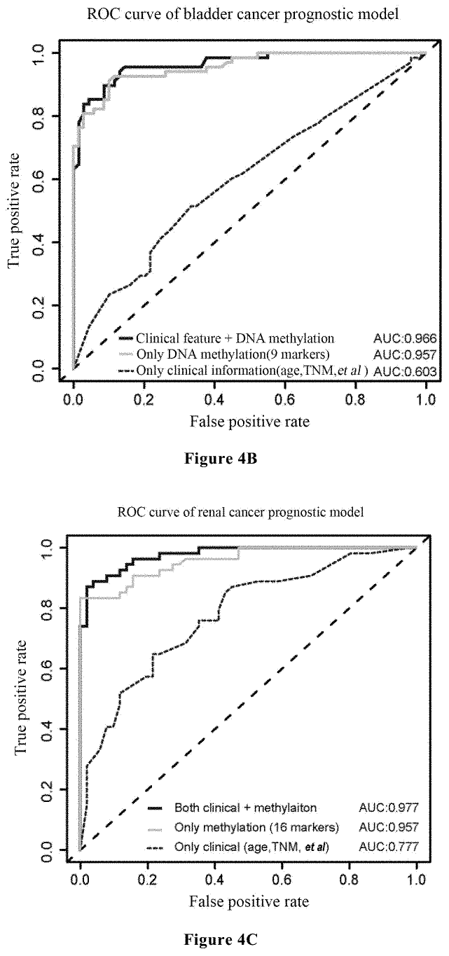

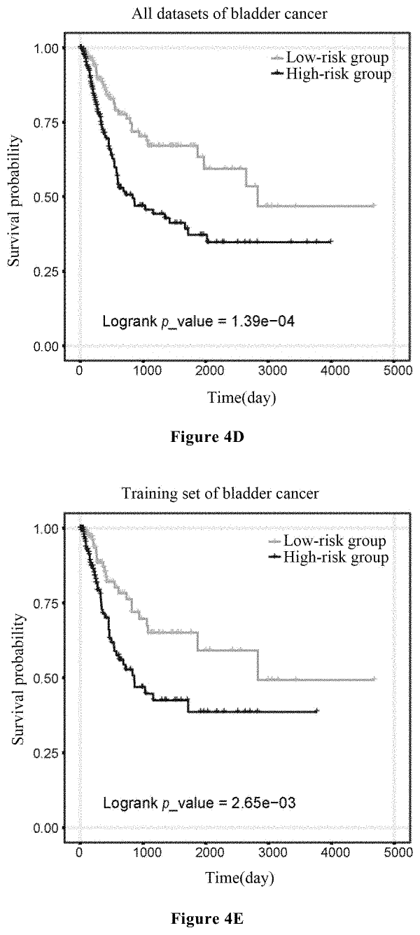

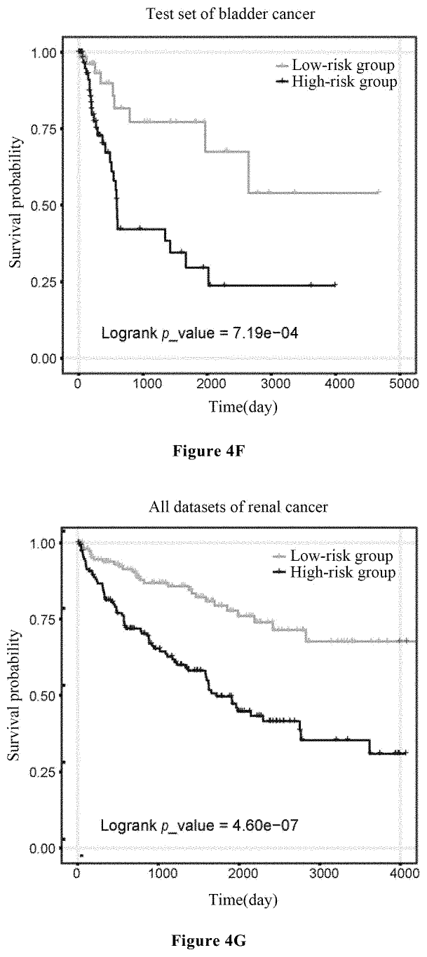

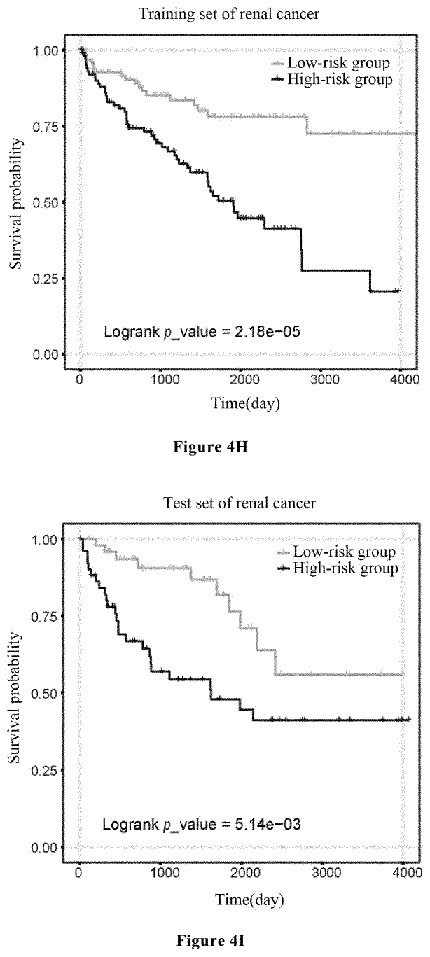

Firstly, a statistical test was used to find the MHBs that can not only distinguish the tumor tissue from the corresponding pericarcinomatous tissue in the available clinical data of TCGA, but also distinguish the aforementioned 313 tumor patients from the healthy people in the urine sediment gDNAs. The specific procedure was shown in FIG. 4A. TCGA 450 K methylation data and urine sediment BS-seq data (results obtained in Example 4) were used for analysis. If the p value of a statistical test in the former was significant, it represented that there was a difference between the tumor tissue and the corresponding pericarcinomatous tissue. If the p value of a statistical test in the latter was significant, it represented that the tumor patients and healthy people can be distinguished by urine sediment gDNAs. By identifying the overlapped regions, regions indicating both of the differences could be found.

These regions were then subjected to univariate and multivariate cox regression analysis. A statistically significant MHBs were selected for LASSO cox prognostic risk assessment to determine high-risk and low-risk groups and a combination of optimal prognostic risk features (resulting in a prognostic risk assessment model). The random forest algorithm was further used for these features, and the features were gradually kicked out until the accuracy of the prognostic model no longer increased. The MHBs (9 MHBs for the prognosis of bladder cancer and 16 MHBs for the prognosis of renal cancer) closely related to the prognosis of bladder cancer and renal cancer were finally found, which can potentially be applied to prognostic survival analysis of tumor patients.

The R packages used in the selection of model features include survival, survminer, glmnet and glmSparseNet. After the features for constructing a model were selected, there were many relevant R packages in R that can be used to analyze ROC curve and K-mean survival. For example, in the Example, the R package used in constructing the ROC curve was ROCR and the R package used in analyzing the K-mean survival was glmSparseNet.

The markers for bladder cancer and renal cancer prognosis were shown in Tables 11 and 12 below.

TABLE 11
Markers for Bladder Cancer Prognosis (9 MHBs)

| | Starting | Termination | | |
|---|---|---|---|---|
| Chromosome | Site | Site | Importance | Type |
| chr10 | 30720672 | 30720759 | 13.09451 | MHB |
| chr10 | 45914483 | 45914559 | 8.876548 | MHB |
| chr19 | 35607208 | 35607231 | 7.932678 | MHB |
| chr1 | 44031286 | 44031306 | 17.51692 | MHB |
| chr21 | 38076854 | 38076871 | 43.3302 | MHB |
| chr21 | 38077596 | 38077665 | 49.92176 | MHB |
| chr2 | 43398069 | 43398085 | 9.750758 | MHB |
| chr2 | 88990993 | 88991089 | 10.95681 | MHB |
| chr2 | 234847745 | 234847792 | 43.62419 | MHB |

TABLE 12
Markers for Renal Cancer Prognosis (16 MHBs)

| | Starting | Termination | | |
|---|---|---|---|---|
| Chromosome | Site | Site | Importance | Type |
| chr10 | 101281679 | 101281743 | 8.484985 | MHB |
| chr11 | 70257148 | 70257258 | 3.651553 | MHB |
| chr13 | 44588054 | 44588213 | 5.223878 | MHB |
| chr14 | 95403135 | 95403150 | 2.406506 | MHB |
| chr14 | 95693820 | 95693832 | 3.274108 | MHB |
| chr15 | 42749747 | 42749885 | 12.2734 | MHB |
| chr17 | 63053928 | 63053939 | 4.037518 | MHB |
| chr17 | 64640443 | 64640600 | 3.395518 | MHB |
| chr19 | 3398705 | 3398743 | 7.070373 | MHB |
| chr19 | 6476950 | 6477038 | 14.66869 | MHB |
| chr1 | 2139220 | 2139296 | 2.998077 | MHB |
| chr1 | 2979310 | 2979346 | 17.31798 | MHB |
| chr1 | 25257913 | 25257952 | 41.67372 | MHB |
| chr1 | 26070245 | 26070333 | 13.778 | MHB |
| chr1 | 156405917 | 156405949 | 3.188925 | MHB |
| chr20 | 524253 | 524414 | 12.52772 | MHB |

The AUC value of the ROC curve of the prognostic survival model constructed by the present inventors was very high (FIG. 4B-4C), especially 0.97 for renal cancer and 0.96 for bladder cancer. The combination of methylation and clinical data (age, TNM, stage, i.e., age, TNM stage, and grading) can optimize prognostic model performance (in the process of modeling, the corresponding clinical variable information such as age, TNM, or stage was integrated into a modeling matrix for modeling). Accordingly, the model constructed by the inventors showed significant differences in survival between high-risk and low-risk groups at the overall level, training set level and test set level (p value<0.05) (FIG. 4D-4I).

The above experimental results showed that the present inventors have developed, for the first time, a model for the diagnosis, localization and prognosis of urogenital tumors that integrates the methylation haplotype and copy number information of urine sediment genomic DNAs. The model can be used to not only predict with high accuracy whether an unknown sample is a tumor or healthy, but also determine the tissue origin of the tumor if the sample is a tumor. By comparing the multivariate classifier algorithms, the GUseek system constructed by the inventors is significantly superior to other commonly used machine algorithm models, including SVM, LASSO, LDA, knn, RandomForest, and Bayes algorithms (FIG. 3B). The prognostic risk assessment model constructed by the present inventors can be potentially applied to survival prognostic assay in tumor patients.

### Example 10: Diagnostic Example

On the first day, the test subjects were enrolled, and a 50 ml of urina sanguinis collection tube was distributed to each subject. The test subjects were then required to collect 50 ml of urina sanguinis in the following morning and send it to the urine collection site of the clinic. The urine was then centrifuged to obtain the corresponding urine sediment. Next, the urine sediment DNAs were extracted and a WGBS library was constructed and sequenced to obtain data information of the F5 features in WGBS. For example, MHL values corresponding to the F5 features in WGBS were calculated using MONOD2 software, and copy number variation data corresponding to the F5 features in WGBS were calculated by using Varbin. The basic protocols can follow those in the above Examples 1-4 and Example 7.

The acquired data information of the F5 features in WGBS was then imported into the classifier model constructed according to Example 7 or 8 of the present application. The model can output a possible category of an unknown subject, such as healthy or unhealthy, in particular which type of tumor it is where the subject is unhealthy. If a patient has developed a tumor and undergone surgery, testing at this time was similar to regular follow-up of the patient after surgery.

### Example 11: Example of Prognosis Assessment

The prognosis model is only for tumor patients. The tumor patients with good prognosis and survival are expressed as a low-risk group, and the tumor patients with poor prognosis and survival are expressed as a high-risk group. The purpose of the prognostic model of the present application is to divide the high-risk and low-risk groups of patients.

On the first day, the test patients with renal or bladder cancer were enrolled, and a 50 ml of urina sanguinis collection tube was distributed to each patient. The test subjects were then required to collect 50 ml of urina sanguinis in the following morning and send it to the urine collection site of the clinic. The urine was then centrifuged to obtain the corresponding urine sediment. Next, the urine sediment DNAs were extracted and sent to a company to measure the 450 K or 850 K chip data of the sample. The data information of the prognostic marker characteristics in Table 11 and/or Table 12 in the 450 K or 850 K chip data was then obtained, such as the corresponding β mean (the mean of probe signals, which is positively correlated with the methylation level) of the prognostic markers in Table 11 and/or Table 12 in the 450 K or 850 K chip data. The acquired data information of the feature candidate prognostic markers in the 450 K or 850 K chip was then imported into the prognostic risk assessment model constructed in Example 9 of the present application. The model can output a possible category of a patient with unknown risk category, such as a high-risk group or a low-risk group. If a patient has developed a tumor and undergone surgery, testing at this time was similar to regular follow-up of the patient after surgery.

Although specific embodiments of the present application have been described in detail, a person skilled in the art will appreciate that various modifications and substitutions can be made to those details from the teachings of the disclosure, all of which are within the scope of the present application. The full scope of the present application is covered by the appended claims and any equivalents thereof.

1. A DNA classification method, comprising:
calculating the MHL value or β mean of a DNA methylation haplotype block of a sample of interest and/or calculating the DNA copy number variation data of the sample of interest; and
calculating the similarity between the MHL value or β mean of the DNA methylation haplotype block of the sample of interest and the MHL value or β mean of a DNA methylation haplotype block of a respective classification label, and/or calculating the similarity between the copy number variation data of the sample of interest DNA and the DNA copy number variation data of a respective classification label; and
determining a classification for the DNA in the sample of interest by using a classifier model and based on the similarity.

2. The method according to claim 1, wherein determining the classification for the DNA in the sample of interest comprises
determining, using a random forest model and based on the similarity, a correlation between the MHL value of the DNA methylation haplotype block of the respective classification label and a human urogenital tumor, and/or a correlation between the DNA copy number variation data of the respective classification label and a human urogenital tumor; and
determining the classification for the DNA in the sample of interest using the classifier model and based on the correlation.

3. The method according to claim 2, wherein
determining the correlation between the MHL value of the DNA methylation haplotype block of the respective classification label and the human urogenital tumor comprises, based on the correlation, ranking the MHL value of the DNA methylation haplotype block to form a vector sequence, and inputting the vector sequence into the random forest model to determine the correlation between the MHL value of the DNA methylation haplotype block and the human urogenital tumor;
and/or
determining the correlation between the DNA copy number variation data of the respective classification label and the human urogenital tumor comprises, based on the correlation, ranking the DNA copy number variation data to form a vector sequence, and inputting the vector sequence into the random forest model to determine the correlation between the DNA copy number variation data of the classification label and the human urogenital tumor.

4. The method according to claim 3, wherein the human urogenital tumor is any one, any two, or all three selected from the group consisting of prostate cancer, urothelial cancer, and renal cancer;
preferably, the renal cancer is a kidney renal clear cell carcinoma,
preferably, the urothelial cancer is upper tract urothelial cancer and/or bladder cancer,
preferably, the prostate cancer is prostate adenocarcinoma; and
preferably, the human urogenital tumor is diagnosed by biopsy from a surgery.

5. The method according to claim 4, wherein the random forest model includes at least three random forest binary classifiers and is selected from any one, any two, any three or all four of the following groups I-VI:
(I). normal-vs-renal cancer, normal-vs-urothelial cancer, and normal-vs-prostate cancer;
(II). renal cancer-vs-normal, renal cancer-vs-urothelial cancer, and renal cancer-vs-prostate cancer;
(III). urothelial cancer-vs-normal, urothelial cancer-vs-renal cancer, and urothelial cancer-vs-prostate cancer; and
(IV). prostate cancer-vs-normal, prostate cancer-vs-renal cancer, and prostate cancer-vs-urothelial cancer.

6. The method according to claim 5, comprising voting for each group, and determining the group with the highest number of votes as the final classification, wherein if equal numbers of votes occur, the category with the highest prediction probability among the groups with the equal number of votes is determined as the final classification.

7. The method according to claim 1, wherein the sample is a urine sample, preferably urina sanguinis, more preferably urine sediment of urina sanguinis.

8. The method according to claim 1, wherein the MHL value of the DNA methylation haplotype block of the sample of interest, the MHL value of the DNA methylation haplotype block of the respective classification label, the DNA copy number variation data of the sample of interest, and the DNA copy number variation data in the respective classification label are all calculated from the sequencing data of the DNAs in a urine sample;
preferably, the DNAs in the urine sample are urine sediment DNAs; and
preferably, the sequencing data is whole genome methylation sequencing data, such as whole genome bisulfite sequencing data; preferably, the sequencing depth is 1×-5×.

9. The method according to claim 1, wherein
the DNA methylation haplotype block of the sample of interest is the same as the DNA methylation haplotype block of the respective classification label; and/or
the DNA copy number variation regions of the sample of interest are the same as the DNA copy number variation regions of the respective classification label;
preferably, the methylation haplotype blocks and the copy number variation regions are those as shown in any one, any two, any three, any four, any five or all six of Tables 1-6, or as shown in Table 11 and/or Table 12.

10. The method according to claim 1, wherein
the MHL value of the DNA methylation haplotype block of the sample of interest and the MHL value of DNA methylation haplotype block of the respective classification label are calculated by using MONOD2 software, and/or DNA copy number variation data of the sample of interest and DNA copy number variation data of the respective classification label are calculated by using Varbin;
preferably, the MHL value corresponding to the respective methylation haplotype block in the WGBS data is calculated by using MONOD2 software, and/or the copy number variation data corresponding to the respective copy number variation region in the WGBS data is calculated by using Varbin, wherein the methylation haplotype block and the copy number variation region are those as shown in any one, any two, any three, any four, any five, or all six of Table 1-6, or as shown in Table 11 and/or Table 12.

11. A method for the detection, diagnosis, classification, risk assessment or prognostic assessment of a human urogenital tumor, comprising
(1) obtaining a urine sample and extracting urine sediment DNAs;
(2) fragmenting the DNAs into fragments of 300-500 bp;
(3) constructing a whole genome library, preferably a whole genome methylation sequencing library, such as a whole genome bisulfate sequencing library, using the obtained DNA fragments; and
(4) classifying the DNA fragments in the library using the method of claim 1, wherein the DNA fragments serve as the DNA in the sample of interest.

12. The method according to claim 11, wherein the urogenital tumor is one or more selected from the group consisting of prostate cancer, urothelial cancer, and renal cancer; and preferably, the renal cancer is kidney renal clear cell carcinoma, the urothelial cancer includes upper tract urothelial cancer and bladder cancer, and the prostate cancer is prostate adenocarcinoma.

13. The method according to claim 11, wherein in step (1), the urine sample is urina sanguinis; and preferably, the urine sample is urine sediment of the urina sanguinis.

14. The method according to claim 11, wherein in step (2), the DNAs are fragmented into fragments of 350-450 bp.

15. (canceled)

16. A device for the detection, diagnosis, classification, risk assessment or prognostic assessment of a human urogenital tumor, comprising
a memory; and
a processor coupled to the memory;
wherein program instructions which can be executed by the processor are stored in the memory, and the program instructions include any one, any two, any three, or all four decision units selected from the group consisting of
I. ‘normal decision unit’:
normal-vs-renal cancer, normal-vs-urothelial cancer, and normal-vs-prostate cancer;
II. ‘renal cancer decision unit’:
renal cancer-vs-normal, renal cancer-vs-urothelial cancer, and renal cancer-vs-prostate cancer;
III. ‘urothelial cancer decision unit’:
urothelial cancer-vs-normal, urothelial cancer-vs-renal cancer, and urothelial cancer-vs-prostate cancer;
IV. ‘prostate cancer decision unit’:
prostate cancer-vs-normal, prostate cancer-vs-renal cancer, and prostate cancer-vs-urothelial cancer;
wherein each decision unit comprises three random forest binary classifiers.

17. The device according to claim 16, wherein the processor is configured to perform a classification method based on instructions stored in the memory, said classification method comprising:
calculating the MHL value or β mean of a DNA methylation haplotype block of a sample of interest and/or calculating the DNA copy number variation data of the sample of interest; and
calculating the similarity between the MHL value or β mean of the DNA methylation haplotype block of the sample of interest and the MHL value or β mean of a DNA methylation haplotype block of a respective classification label, and/or calculating the similarity between the copy number variation data of the sample of interest DNA and the DNA copy number variation data of a respective classification label; and
determining a classification for the DNA in the sample of interest by using a classifier model and based on the similarity.

18. The device according to claim 16, wherein the urogenital tumor is one or more selected from the group consisting of prostate cancer, urothelial cancer, and renal cancer;
preferably, the renal cancer is a kidney renal clear cell carcinoma,
preferably, the urothelial cancer is upper tract urothelial cancer and/or bladder cancer, and
preferably, the prostate cancer is prostate adenocarcinoma.

19-21. (canceled)
